SEQUENCE LISTING (6 sequences)

SEQ ID NO: 1 - DNA, 596 bp, Aspergillus fumigatus
  CDS (14)..(101): n = a, c, g or t/u
tccgtaggtgaacntgcggaaggatcattacngagtgagggccttntgggtccaacttcccaccngtgtctatcgtaccttgttgcttcggcgggcccgcngtttcgacggccgccggggaggcttgcgcccccgggcccgcgcccgccgaagaccccaacatgaacgctgttctgaaagtatgcagtctgagttgattatcgtaatcagttaaaactttcaacaacggatctcttggttccggcatcgatgaagaacgcagcgaaatgcgataagtaatgtgaattgcagaattcagtgaatcatcgagtctttgaacgcacattgcgccccctggtattccggggggcatgcctgtccgagcgtcattgctgccctcaagcacggcttgtgtgttgggcccccgtccccctctcccgggggacgggcccgaaaggcagcggcggcaccgcgtccggtcctcgagcgtatggggctttgtcacctgctctgtaggcccggccggcgccagccgacacccaactttatttttctaaggttgacctcggatcaggtagggatacccgctgaacttaagcatatcaataagcggagga

SEQ ID NO: 2 - DNA, 16 bp, Artificial Sequence
  Description of Artificial Sequence  Synthetic Primer
ttggttccggcatcga

SEQ ID NO: 3 - DNA, 17 bp, Artificial Sequence
  Description of Artificial Sequence  Synthetic Primer
gcagcaatgacgctcgg

SEQ ID NO: 4 - DNA, 25 bp, Artificial Sequence
  Description of Artificial Sequence  Synthetic Primer
tgaagaacgcagcgaaatgcgataa

SEQ ID NO: 5 - DNA, 552 bp, Fusarium sp.
tctccgttggtgaaccagcggagggatcattaccgagttatacaactcccaaacccctgtgaacttacctataacgttgcttcggcgggaaaccgacggccccgtaagacgggccgcccccgccagaggaccccctaactcttgttatatcctgtttctctgagtacaacaagcaaataaattaaaactttcaacaacggatctcttggctctggcatcgatgaagaacgcagcgaaacgcgataagtaatgtgaattgcagaatttagtgaatcatcgaatctttgaacgcacattgcgcccgccagtattctggcgggcatgcctgtccgagcgtcattacaaccctcaggcccccgggcctggtgttggggatcggtcggagccctccggggcacacgccgtcccccaaattcagtggcggtctcgctgcagcctccattgcgtagtagctaacacctcgcaactggaaggcggcgcggccacgccgtaaaacccccccaacttctgaatgttgacctcggatcaggtaggaatacccgctgaacttaa

SEQ ID NO: 6 - DNA, 495 bp, Scedosporium Prolificans
ccgagttattactccaaaccctttgtgaaccttacctatgttctgttgcctcggcggggaggaagaccccttaaaaagggcccagcccccgccggcagcaccaaactcttgcatttatagcggattacatttctgaatacaatacaaaacaaatcaaaactttcaacaacggatctcttggttctggcatcgatgaagaacgcagcgaaatgcgataagtaatgtgaattgcagaattcagtgaatcatcgaatctttgaacgcacattgcgccggcagtaatctgccggcatgcctgtccgagcgtcatttcaaccctcgagcctaggtttttacaagcccaaggatcggtgttggggcgctacggttgtcatctgaccgccgtaggctctgaaatacagtggcggtcccgccgcggcgccttctgcgtagtagatttacaactcgcattgggtcccggcgaaggccagccgtcaaaccctctattcttatggt

(19) United States
(12) Patent Application Publication
(10) Pub. No.: US 2005/0048509 A1
(43) Pub. Date: Mar. 3, 2005

(54) Diagnosis of invasive mold infection

(76) Inventors: Xiang-Yang Han, Bellaire, TX (US); Jeffrey J. Tarrand, Houston, TX (US); Audrey S. Pham, Houston, TX (US); Gregory S. May, Houston, TX (US)

Correspondence Address: FULBRIGHT & JAWORSKI L.L.P., 600 CONGRESS AVE., SUITE 2400, AUSTIN, TX, 78701, US

(21) Appl. No.: 10/672,300

(22) Filed: Sep. 26, 2003

Related U.S. Application Data
(60) Provisional application No. 60/414,008, filed on Sep. 27, 2002.

(51) Int. Cl. C12Q 1/68

(52) U.S. Cl. 435/6

(57) ABSTRACT

The present invention provides methods for detecting the presence of invasive pathogenic molds in biological samples that are based on amplification of mold nucleic acids. The methods may further comprise quantitating and real time detection of the invasive mold. The methods of the invention are highly specific and do not co-amplify human or other yeast nucleic acids. The methods of the invention are also extremely sensitive. Thus, methods for diagnosing infections caused by invasive mold are provided. The invention also provides kits for detection of invasive molds.

### BACKGROUND OF THE INVENTION

The present application claims priority to co-pending U.S. Provisional Application, Ser. No. 60/414,008 filed Sep. 27, 2002. The entire text of the above-referenced disclosure is specifically incorporated herein by reference without disclaimer.

1. Field of the Invention

The present invention relates generally to the fields of microbiology and pathology. More particularly, it concerns the development of methods to diagnose invasive mold infections using real-time PCR™ based methods.

2. Description of Related Art

Aspergillus and other septate molds are ubiquitous and may cause invasive aspergillosis (IA) or invasive mold infection (IMI) among patients with neutropenia and immunosuppression. These infections carry a fatality rate of 92% (Paterson and Singh, 1999). Patients undergoing hematopoietic stem cell transplantation are particularly vulnerable to this infection with an estimated incidence of 6.4% by patient (Paterson and Singh, 1999). Although nationwide incidence of the disease is unknown, the number of cases has been increasing over the past decade largely due to an increase of the susceptible populations (Latge, 1999).

Definitive and early diagnosis of IMI is crucial for proper patient management, clinical research, and epidemiological studies. A number of factors, however, hamper this effort. First, definitive diagnosis entails tissue sampling by invasive procedures, which is frequently impractical due to associated risks, particularly thrombocytopenia. The diagnosis is often rendered late in the disease course. In fact, many such cases are established by autopsy (Vogeser et al., 1997; Kontoyiannis et al., 2000). Second, immunocompromized patients may be unable to mount an effective immune response, which precludes an antibody-based diagnosis (Latge, 1999). Third, many molds, particularly Aspergillus, are rarely isolated from blood cultures (Tarrand et al., 2000), unlike bacteria. Fourth, isolation from solid tissue is also infrequent for Aspergillus (Tarrand et al., 2000). On the other hand, isolation of molds from a normal host's airway is not uncommon due to its ubiquitous nature, and in an immunocompromised patient, this may cause confusion between active disease, colonization, or contamination. Consequently, a clinical diagnosis of “probable” or “possible” IMI is frequently necessary, made through a correlation of clinical manifestation, radiological findings, microbiological culture, and exclusion of other etiologies (Ascioglu et al., 2002). Therefore, a less invasive, reliable, sensitive, and specific diagnostic test for IMI is urgently needed to solve this diagnostic ambiguity.

Detection of circulating Aspergillus DNA in the bloodstream by PCR™ has been emerging as a promising diagnostic approach in recent years (Bretagne et al., 1998; Einsele et al., 1997; Hebart et al., 2000; Van Burik et al., 1998; Williamson et al., 2000; Yamakami et al., 1996). These studies have shown that PCR™ has the advantages of higher sensitivity, less environmental contamination, and easier and repeatable sampling of blood. However, these studies are all limited by smaller patient populations, possible subjectivity, cross-reactivity to Candida, lack of quantitation, or a combination of all these factors. Thus, the art still lacks a reliable method for detecting and diagnosing invasive mold infections (IMI).

### SUMMARY OF THE INVENTION

With these and other improvements in mind, the present inventors have developed detection methods based on the amplification of invasive mold DNA using primers that specifically and selectively amplify invasive mold DNA. In some embodiments, real-time PCR-based methods are described that combine amplification and simultaneous probe hybridization to achieve sensitive, specific, and quantitative detection of infectious molds in real time thereby providing instant detection of invasive molds.

The present invention therefore provides methods for detecting the presence of an invasive mold in a subject comprising identifying 5.8S ribosomal RNA of an invasive mold or a DNA encoding the ribosomal RNA in a sample obtained from the subject. In other embodiments, the present invention comprises mixing the sample with primers that hybridize to the 5.8S ribosomal RNA of an invasive mold; amplifying the sample comprising the RNA; and determining the presence or absence of an amplification product in the sample obtained wherein the presence of an amplification product is indicative of the presence of an invasive mold. In particular embodiments the primers hybridize to the 5.8S ribosomal or DNA encoding the RNA under high stringency conditions as are disclosed herein.

In some embodiments, the methods further comprise quantitating the amplification product whereby the amount of invasive mold nucleic acid is quantitated. In some aspects the quantitating comprises: a) mixing a first probe capable of hybridizing to a nucleic acid sequence of the invasive mold; b) mixing a second probe capable of hybridizing to a standard nucleic acid that is amplified to a pre-determined quantity in the same amplification reaction; c) comparing or quantifying the amount of the first probe to the amount or quantity of the second probe. The mixing of the first and second probes occur during the linear phase of the amplification.

In some specific embodiments, the first probe comprises nucleic acids that hybridize to the nucleic acid sequence of SEQ ID NO:1, SEQ ID NO:5 or SEQ ID NO:6 or fragments thereof. In other specific embodiments, the first probe comprises the sequence 5′-TGAAGAACGCAGCGAAATGCGATAA-3′ (SEQ ID. NO:4).

In some aspects of this method, it is contemplated that the probe will be labeled for detection purposes. Several types of labels are known in the art including fluorescent labels, radioisotopes, colorimetric labels, ligands, antibodies, enzymatic tags and the like. In some specific embodiments, the use of fluorescent labels is particularly contemplated. Some non-limiting examples of fluorescent labels include 6-carboxyfluorescein (FAM), 6-carboxy-N,N,N′,N′-tetramethylrhodamine (TAMRA), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, or Texas Red. In specific aspects, the probe comprises the sequence 5′-6-FAM-TGAAGAACGCAGCGAAATGCGATAA-TAMRA-3′ (SEQ ID NO:4).

It is contemplated that the nucleic acid from the sample may be RNA or DNA. In embodiments where the nucleic acid is RNA, the amplification reaction is preceded by a reverse transcription reaction.

The invasive molds that are detected by the methods of the invention include those of the Aspergillus species, the Fusarium species, and/or the Scedosporium species. Some non-limiting examples of invasive molds of the Aspergillus species include Aspergillus fumigatus, Aspergillus flavus, Aspergillus terreus, Aspergillus vesicularis, Aspergillus nidulans, or Aspergillus niger. Non-limiting examples of invasive molds of the Fusarium species include Fusarium solani and those of the Scedosporium species include Scedosporium prolificans.

In particular embodiments, the present invention contemplates the use of samples such as i.e., biological samples and/or nucleic acid containing samples. Samples are described in detail in the specification and may comprise serum, blood, plasma, cells, tissues, aspirates, biopsies, fine needle aspirates, skin biopsies, lymph fluid or urine.

In some embodiments, primers are comprised of nucleic acids that hybridize to the nucleic acid sequence comprised in SEQ ID NO: 1 or fragments thereof. In some specific aspects of this embodiment, the primers comprise the nucleic acid sequence TTGGTTCCGGCATCGA (SEQ ID. NO:2) or GCAGCAATGACGCTCGG (SEQ ID. NO:3).

In other embodiments, the primers are comprised of nucleic acids that hybridize to the nucleic acid sequence comprised in SEQ ID NO: 5 or fragments thereof. In yet other embodiments, the primers are comprised of nucleic acids that hybridize to the nucleic acid sequence comprised in SEQ ID NO: 6 or fragments thereof.

The amplification may be carried out by polymerase chain reaction (PCR™). However, other forms of amplification known in the art may be used as well. A brief description of PCR™ and certain other amplification reactions is provided infra in the specification.

In some embodiments of the methods, identifying the presence of invasive mold is in real time. The methods of the invention are extremely sensitive with a detection range of 200 fg to 20 ng of DNA. The methods of the invention are highly specific and do not amplify or detect human or candidal DNA.

In some embodiments, the methods further comprise obtaining a sample such as a biological or nucleic acid containing sample from a subject. In other embodiments, the methods further comprise isolating nucleic acids from the biological sample.

The invention also provides kits for detecting an invasive mold in a biological sample. Such kits comprise: a) primers that hybridize to the 5.8S ribosomal RNA of an invasive mold or a DNA encoding the RNA; and b) reagents for an amplification reaction comprising a heat-stable DNA polymerase enzyme, buffers, water, magnesium chloride, and deoxynucleotides; each enclosed in a suitable container means.

In some embodiments, the kit may comprise primers comprising nucleic acids that hybridize to the nucleic acid sequence of SEQ ID NO:1, SEQ ID NO:5 or SEQ ID NO:6 or fragments thereof. In other embodiments, primers of the kit comprise the nucleic acid sequence 5′-TTGGTTCCGGCATCGA-3′ (SEQ ID. NO:2) or 5′-GCAGCAATGACGCTCGG-3′ (SEQ ID. NO:3).

In other embodiments, the kits may further comprising one or more probes that hybridize to the 5.8S ribosomal RNA of the invasive mold or fragments thereof, or a DNA encoding the RNA. It is contemplated that these probes will comprise a nucleic acid sequence that hybridizes to the nucleic acid sequence of SEQ ID NO:1, SEQ ID NO:5 or SEQ ID NO:6 or fragments thereof. In specific aspects, the kit will comprise one or more probes comprising the sequence 5′-TGAAGAACGCAGCGAAATGCGATAA-3′ (SEQ ID. NO:4). In yet other aspects, the probe is labeled with one or more suitable detectable label(s). Alternatively, labels may be provided with instructions on how to label the probe(s) with the label.

The kits of the invention may also comprise reagents to isolate nucleic acids from a sample, such as a biological sample and/or a nucleic acid containing sample. In some embodiments, reagents used to isolate mRNA will be comprised in such a kit. In other embodiments, reagents used to isolate DNA will be comprised in such a kit.

It is also contemplated that the kits will comprise suitable standards that may be amplified and/or detected and/or quantified. This will include both negative and positive standards as known in the art.

As used herein the specification, “a” or “an” may mean one or more. As used herein in the claim(s), when used in conjunction with the word “comprising”, the words “a” or “an” may mean one or more than one. As used herein “another” may mean at least a second or more.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-1B. Detection of mold (Aspergillus) DNA by real-time PCR. FIG. 1A. Detection signal (ΔRₙ) and PCR cycles versus the quantity of Aspergillus DNA: (●) 0 fg, (∘) 2×10² fg, (▪) 2×10³ fg, (□) 2×10⁴ fg; (▾) 2×10⁵ fg, and (X) 2×10⁷ fg. Horizontal line represents the threshold of detection cycle. FIG. 1B. A semi-log standard curve of FIG. 1A. from another experiment. C_{T}=detection cycles. (●)=Aspergillus DNA standard. (∘)=serum being tested.

FIG. 2. Quantities of mold DNA in 559 sera from patients with “documented”, “probable”, “possible”, and “unlikely” IMI tested by real-time PCR. Signals below the negative control (equivalent to 10 fg) were given an arbitrary quantity between 1 to 10 fg for the purpose of plotting and logarithmic conversion.

### DESCRIPTION OF ILLUSTRATIVE EMBODIMENTS

Invasive mold infections (IMI) that infect predominantly immunocompromised patients have poor outcome and present a diagnostic challenge. For example, the detection of Aspergillus infection has been difficult as blood, urine or cerebrospinal fluid cultures are rarely positive, although, the fungi can be seen in smears and biopsies from infected tissue. Thus, typical diagnostic methods more than often require invasive or surgical procedures that would cause additional detrimental effects to the already immunocompromised patient. The present inventors have developed diagnostic assays based on PCR™ to diagnose IMI by detecting mold nucleic acids in the serum. In some embodiments, the methods are quantitative real-time PCR-based. Thus, the present invention provides methods to detect and diagnose pathogenic molds that cause IMI including those of the Aspergillus species, Fusarium species and Scedosporium species in biological samples such as serum, plasma, blood, or other body fluids that can be easily obtained and therefore provide a significant advantage in the early detection and treatment regimen of such infections. Assays of the present invention are extremely sensitive and can detect 200 fg to 20 ng (5-log range) mold DNA and allows a cutoff of 110 fg which is equivalent to that of 3 genomes in the case of molds of the Aspergillus species, Fusarium species and Scedosporium species. The methods are also highly specific in selectively amplifying and identifying the mold DNA as evidenced by the failure to co-amplify or detect human or candidal DNA.

Cases of IMI are defined in the art as “definitive or documented”, “probable”, “possible”, and “unlikely” IMI. A detailed description of each of these terms is set forth in sections below. The specificity of the present methods is also demonstrated by negative results in 35 patients (76 sera) with “unlikely IMI” at the cutoff. For patients with “possible”, “probable,” and “documented IMI” that were diagnosed by a combination of clinical, microbiological, and histological criteria, this real-time PCR™ based method showed positivity in 40% (12 of 30), 68% (19 of 28), and 85% (11 of 13) cases, respectively, upon testing of multiple serum samples. The overall serum positivity rate for these IMI patients was 15% (73 of 483). Quantitative analysis of the positive sera estimated the bodily circulating mold burden to be 1.6×10⁵ genomes (5.3 ng) by geometric mean with 4.2×10⁷ genomes (1,400 ng) being the highest. These results indicate that the diagnostic methods of the present invention are clearly preferable to the existing IMI diagnostic methods in the art in that they are superior in diagnostic potential as well as are non-invasive and provide a rapid diagnosis. The present invention also contemplates kits for the detection of IMI based on the methods described herein.

### A. Invasive Molds and Infections

Infections by invasive fungal pathogens are responsible for the mortality of patients that are immunosuppressed including, patients afflicted with hematological cancers, post-operative patients, transplant patients, those infected by HIV, those undergoing chemotherapy, and cancer patients receiving immunosuppressive medications. As discussed in a recent review by Brakhage and Langfelder (2002), species of the Aspergillus family account for majority of these fungal infections. As the art lacks specific diagnostic methods, invasive aspergillosis alone is associated with a high mortality rate that ranges from 30% to 90% (Brakhage and Langfelder, 2002). Molds of the Fusarium species and Scedosporium species are also responsible for severe infections that are associated with mortality in immunosuppressed patients (Oliveria et al., 2002; Baddley et al., 2001).

Aspergillosis is the most common mold infection in immunosuppressed patients. Aspergillosis is a term that encompasses a variety of disease processes caused by Aspergillus species. Aspergillus species are ubiquitous; their spores are constantly being inhaled. Of the more than 300 species known, only some are ordinarily pathogenic for man and these include: A. fumigatus, A. flavus, A. niger, A. nidulans, A. terreus, A. sydowi, A. flavatus, A. glaucus, and A. vesicularis. Aspergillosis is increasing in prevalence and is particularly a problem among patients with chronic respiratory disease or immunocompromised patients. Opportunistic pulmonary aspergillosis is characterized by widespread bronchial erosion and ulceration, followed by invasion of the pulmonary vessels, with thrombosis, embolization and infarction. Clinically, infection manifests as a necrotizing patchy bronchopneumonia, sometimes with hemorrhagic pulmonary infarction. In about 40% of cases, there is hematogenous spread to other sites. Aspergillosis is also a rare but devastating complication of traumatic wounds, such as, burn wounds, frost bite wounds, or wounds developed by diabetics, where amputation is often required for cure. Invasive aspergillosis is commonly fatal, so aggressive diagnosis and treatment is required.

### B. Case Definition for IMI

Cases of IMI are defined herein according to the criteria established by EORTC and the Mycoses Study Group (Ascioglu et al., 2002), depending on the degree of diagnostic certainty, the cases are defined as “definitive or documented”, “probable”, “possible”, and “unlikely” IMI. Briefly, “definitive or documented” IMI represent a tissue diagnosis where branched septate hyphae, inflammation, and necrosis are seen microscopically and/or the fungus is successfully cultured from the tissue. Most of these patients have pulmonary IMI and are typically neutropenic and/or immunosuppressed for an extended period of time and in general exhibit prolonged pneumonia unresponsive to anti-bacterial therapy with nodular and/or cavitory lesions in the lung radiologically. Patients with “probable IMI” are also typically immunocompromised and have clinical and radiological features of IMI, and Aspergillus or other septate molds are isolated twice or more from respiratory specimens (such as sputa, bronchoalveolar lavages, and bronchial washings). In some cases of “probably IMI” although pneumonia is seen in autopsy, molds are not observed histologically or cultured microbiologically from the autopsy lung tissue. Such cases are diagnosed as “probable IMI.” Patients with “possible IMI” are those with atypical pulmonary radiology, pneumonia unresponsive to anti-bacterials, and isolation of mold once (rarely without) from the airway. Patients who did not meet the criteria for possible IMI represented “unlikely IMI.”

### C. Biological Samples and Methods of Obtaining Samples

A “biological sample” or “sample” is defined herein as any cell, cellular extract, tissue, organ or bodily fluid. This includes tissue sections, specimens, aspirates, biopsies including bone marrow aspirates, tissue biopsies, tissue swabs, fine needle aspirates and even skin biopsies. Other suitable examples are fluids, including samples where the body fluid is peripheral blood, serum, lymph fluid, cerebrosipnal fluid, seminal fluid or urine. Stools may even be used.

The present invention discloses methods comprising, in part, providing or obtaining samples from a human subject. One specific embodiment of the invention involves collection of a sample of peripheral blood from a human subject. This can be accomplished through intravenous withdrawal of blood or other available means from any exterior limb or other vein comprising part of the peripheral circulatory system.

### D. Isolation of DNA from the Samples

Efficient DNA extraction is crucial for any PCR-based or other amplification assay, particularly to detect microbial DNA from a biological sample. One of skill in the art will appreciate that any DNA isolation method known in the art may be used (for example, DNA isolation methods set forth in Maniatis et al. (1988), incorporated herein by reference). However, in a preferred embodiment, the present invention uses a silica-binding method that is capable of extracting extremely low levels (picogram) of fungal DNA along with microgram levels (10⁶-fold more) of human DNA in a serum sample. Experimental details of the method are described in the section entitled as “Example 1.” This method is superior to the typically used phenol-chloroform methods and some commercial DNA extraction kits. A previous study (Fahle et al, 2000), also reported that the silica-binding method was the best among six methods in extracting cytomegalovirus DNA from serum and cerebrospinal fluid.

The inventors prefer serum to extract mold DNA because it appears to be a better source than plasma or whole blood for the extraction of fungal or bacterial DNA (Yamakami, et al., 1996; Boom, et al., 1990; Bowman et al., 2001; Stynen et al., 1995; Fahle et al., 2000; Zerva et al., 2001). In addition, serum typically has much less human DNA (1-5 g/ml) than whole blood (30-50 g/ml), which might favor the extraction of minute quantities of exogenous microbial DNA.

### E. Isolation and Quantitation of RNA Transcripts from the Sample

Optionally, one may isolate RNA from a biological sample which may then be reverse transcribed to DNA, prior to amplification. Many methods to isolate total cellular RNA are well know to those skilled in the art. See, for example, Chomczynski and Sacchi (1987). A particular method to accomplish this task is the use of the Trizol reagent (Gibco Life Technologies) to extract total cellular RNA. The Trizol procedure involves homogenization of the cells in a blender followed by extraction with the phenol-based Trizol reagent. The RNA is then precipitated with isopropyl alcohol and washed with ethanol before being redissolved in RNAse-free water or 0.5% SDS.

### F. Reverse Transcription

Reverse transcription (RT), is a process for the conversion of mRNA into DNA. Briefly, a poly-dT primer is annealed to the poly-A tail of a messenger RNA. This provides a free 3′ end for extension by reverse transcriptase (RT). The enzyme performs 5′→3′ synthesis, using the mRNA as a template. The intermediate product, a hybrid RNA-DNA molecule, is created. At the end of this reaction, the enzyme “loops back” on itself by using the last few bases of the reverse transcript as a template for synthesis of a complete, i.e., a complementary DNA that displaces the mRNA. This creates a “hairpin” structure. The original mRNA can then be degraded by alkali treatment, producing a single-stranded DNA. The hairpin provides a natural primer for the next step—the use of DNA polymerase I to convert the single-stranded DNA into double-stranded DNA, or a cDNA. The hairpin is removed by S1 nuclease.

Methods of reverse transcribing RNA into cDNA are well known and described in Sambrook et al. (2001). Alternative methods for reverse transcription utilize thermostable, RNA-dependent DNA polymerases. These methods are described in WO 90/07641 filed Dec. 21, 1990.

### G. Amplification Methodology

1. Primers

In general, nucleic acid amplification methodology relies upon the use of primers, which facilitate the amplification process. The word primer, as defined herein, is meant to encompass any nucleic acid that is capable of priming the synthesis of a nascent nucleic acid in a template-dependent process. Typically, primers are oligonucleotides from ten to twenty-five base pairs in length, but longer sequences can be employed. Primers may be provided in double-stranded or single-stranded form, although the single-stranded form is preferred. Specific embodiments of the present invention disclose primers for use in the amplification reactions.

In one embodiment of the present invention, primers were created based on complementarity to the 5.8S ribosomal RNA gene of Aspergillus fumigatus, with GeneBank ID:AF138288, which is a highly conserved gene in the Aspergillus species, for e.g., A. fumigatus, A. flavus, A. terreus, A. niger and others, as well as in certain other infectious molds including those belonging to the Fusarium species and Scedosporium species. Of these, primers corresponding to SEQ ID NO:2 (TTGGTTCCGGCATCGA) or and SEQ ID NO:3 (GCAGCAATGACGCTCGG) have been used in the methods described herein. These sequences were selected to avoid significant homology to and thus amplification of yeast or human DNA. These specific primers were selected by screening of multiple primers for optimum results. Primers representing shortened or lengthened versions of SEQ ID NO:2 and SEQ ID NO:3 are also contemplated.

However, the present invention may be performed using a variety of other suitable primers. For example, one can use primers that are complementary to the gene product of a Fusarium species such as that represented by GenBank ID:AF178419 (SEQ ID. NO:5) which corresponds to F. solani 5.8S ribosomal RNA gene, or a fragment thereof, or that of Scedosporium species such as that represented by GenBank ID: AF022485 (SEQ ID. NO:6), as long as these primers are specific for detecting the infectious organism and do not amplify human or other yeast sequences.

Furthermore, one of skill in the art will also recognize that a primer designed to be complementary to other fragments of the gene described in GeneBank ID:AF138288 may be used as a primer for detecting for the presence of A. fumigatus in a sample as long as this does not co-amplify human or yeast DNA. Alternatively, one could use primers based on the sequence of any other pathogen, including, A. flavus, A. terreus, A. niger, A. vesicularis, A. nidulans, F. chlamydosporum, F. solani and S. prolificans species.

The primers may be prepared by oligonucleotide synthesis according to standard methods. See, for example, Itakura and Riggs (1980). Additionally, U.S. Pat. Nos. 4,659,774, 4,816,571, 5,141,813, 5,264,566, 4,959,463, 5,428,148, 5,554,744, 5,574,146, 5,602,244, each of which is incorporated herein by reference, describe methods of preparing oligonucleotides. In addition, primers are available commercially at affordable rates. Primers may also be synthesized by recombinant methods using products incorporating viral and bacterial promoters available from Promega (Madison, Wis.).

The use of a primer of between 13 and 100 nucleotides, preferably between 17 and 100 nucleotides in length, or in some aspects of the invention up to 1-2 kilobases or more in length, allows the formation of a duplex molecule that is both stable and selective. Molecules having complementary sequences over contiguous stretches greater than 20 bases in length are generally preferred, to increase stability and/or selectivity of the hybrid molecules obtained. One will generally prefer to design nucleic acid molecules for hybridization having one or more complementary sequences of 20 to 30 nucleotides, or even longer where desired. Such fragments may be readily prepared, for example, by directly synthesizing the fragment by chemical means or by introducing selected sequences into recombinant vectors for recombinant production.

In some embodiments, the primers may be labeled as described below for probes or by other methods known in the art.

2. Probes

The invention further utilizes probes for the identification as well as the real-time detection and/or quantitation of invasive mold DNA. As well known in the art, a probe is a nucleic acid molecule that is complementary to a target sequence, which is exemplified in the present invention by a DNA sequence that is specific or unique to an invasive mold. Nucleic acid sequences that are “complementary” are those that are capable of base-pairing according to the standard Watson-Crick complementary rules. As used herein, the term “complementary sequences” means nucleic acid sequences that are “essentially complementary”, as may be assessed by the same nucleotide comparison set forth above, or as defined as being capable of hybridizing to the nucleic acid segment of under relatively stringent conditions such as those described herein. Alternatively the probes may be “substantially complementary”, wherein the sequence of the probe is complementary to 80%, 85%, 90%, 95%, or 99% of an invasive mold DNA sequence. In some non-limiting examples, the probes of the present invention may encode SEQ ID NO:1, SEQ ID NO:5, SEQ ID NO:6 or fragments thereof. In one particular embodiment, a probe of the invention has the sequence TGAAGAACGCAGCGAAATGCGATAA (SEQ ID NO:4).

As probes are used as hybridizing fragments, they should be of sufficient length to provide specific hybridization to a RNA or DNA. The use of a hybridization probe of between about 10-14 or 15-20 and about 100 nucleotides in length allows the formation of a duplex molecule that is both stable and selective. Molecules having complementary sequences over stretches greater than 20 bases in length are generally preferred, in order to increase stability and selectivity of the hybrid, and thereby improve the quality and degree of particular hybrid molecules obtained.

Sequences of 17 bases long should occur only once in the human genome and, therefore, suffice to specify a unique target sequence. Although shorter oligomers are easier to make and increase in vivo accessibility, numerous other factors are involved in determining the specificity of hybridization. Both binding affinity and sequence specificity of an oligonucleotide to its complementary target increases with increasing length. It is contemplated that exemplary oligonucleotides of 8, 9, 10, 11, 12, 13, 14, 15, 16, 17, 18, 19, 20, 25, 30, 35, 40, 45, 50, 55, 60, 65, 70, 75, 80, 85, 90, 95, 100 or more base pairs will be used, although others are contemplated. Longer polynucleotides encoding 250, 300, 500, 600, 700, 800, and longer are contemplated as well.

The probes of the present invention may be made by methods well known in the art, such as chemical synthesis or by recombinant methods as set forth above for primers.

In some embodiments, probes of the present invention may be labeled, such as with fluorescent compounds, radioactive isotopes, antigens, ligands such as biotin-avidin, colorimetric compounds, or other labeling agents known to those of ordinary skill in the art, to allow detection and quantification of amplified DNA. The use of fluorescent labels is especially contemplated in the real-time PCR™ methods of the present invention. Some examples of fluorescent labels include 6-carboxyfluorescein (FAM), 6-carboxy-N,N,N′,N′-tetramethylrhodamine (TAMRA), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, and Texas Red. Alternatively, enzyme tags such as urease, alkaline phosphatase or peroxidase may be used. In the case of enzyme tags, colorimetric indicator substrates are known that can be employed to provide a detection means that is visibly or spectrophotometrically detectable, to identify specific hybridization with complementary nucleic acid containing samples.

3. Hybridization

Accordingly, the primer and probe nucleotide sequences of the invention may be used for their ability to selectively form duplex molecules with complementary stretches of DNAs and/or RNAs or to provide primers for amplification of DNA or RNA from samples or as probes to detect specific sequences in the amplified DNA. Depending on the application envisioned, one would desire to employ varying conditions of hybridization to achieve varying degrees of selectivity of the probe or primers for the target sequence.

For applications requiring high selectivity, one will typically desire to employ relatively high stringency conditions to form the hybrids. For example, relatively low salt and/or high temperature conditions, such as provided by about 0.02 M to about 0.10 M NaCl at temperatures of about 50° C. to about 70° C. Such high stringency conditions tolerate little, if any, mismatch between the probe or primers and the template or target strand and would be particularly suitable for isolating specific genes or for detecting specific mRNA transcripts. It is generally appreciated that conditions can be rendered more stringent by the addition of increasing amounts of formamide.

A medium stringency condition could be provided by about 0.1 to 0.25 M NaCl at temperatures of about 37° C. to about 55° C., while a low stringency condition could be provided by about 0.15 M to about 0.9 M salt, at temperatures ranging from about 20° C. to about 55° C. Hybridization conditions can be readily manipulated depending on the desired results. In other embodiments, hybridization may be achieved under conditions of, for example, 50 mM Tris-HCl (pH 8.3), 75 mM KCl, 3 mM MgCl₂, 1.0 mM dithiothreitol, at temperatures between approximately 20° C. to about 37° C. Other hybridization conditions utilized could include approximately 10 mM Tris-HCl (pH 8.3), 50 mM KCl, 1.5 mM MgCl₂, at temperatures ranging from approximately 40° C. to about 72° C.

In general, it is envisioned that the probes or primers described herein will be useful as reagents in solution hybridization, as in PCR™ or other amplification methods, as well as in embodiments employing a solid phase. In embodiments involving a solid phase, the test DNA (or RNA) is adsorbed or otherwise affixed to a selected matrix or surface. This fixed, single-stranded nucleic acid is then subjected to hybridization with selected probes under desired conditions. The conditions selected will depend on the particular circumstances (depending, for example, on the G+C content, type of target nucleic acid, source of nucleic acid, size of hybridization probe, etc.). Optimization of hybridization conditions for the particular application of interest is well known to those of skill in the art. After washing of the hybridized molecules to remove non-specifically bound probe molecules, hybridization is detected, and/or quantified, by determining the amount of bound label. Representative solid phase hybridization methods are disclosed in U.S. Pat. Nos. 5,843,663, 5,900,481 and 5,919,626. Other methods of hybridization that may be used in the practice of the present invention are disclosed in U.S. Pat. Nos. 5,849,481, 5,849,486 and 5,851,772. The relevant portions of these and other references identified in this section of the specification are incorporated herein by reference.

4. Amplification by PCR™

A number of template dependent processes are available to amplify the pathogenic mold DNA in a given biological sample as described herein. One of the best known amplification methods in the polymerase chain reaction (referred to as PCR™) which is described in detail in U.S. Pat. Nos. 4,683,202 and 4,800,159, and in Innis et al., 1990. Briefly, in PCR™, two primer sequences are prepared that are complementary to regions on opposite complementary strands of the marker sequence. An excess of deoxynucleoside triphosphates are added to a reaction mixture along with a heat-stable DNA polymerase, e.g., Taq polymerase or Vent polymerase. If the marker sequence is present in a sample, the primers will bind to the marker and the polymerase will cause the primers to be extended along the marker sequence by adding on nucleotides. By raising and lowering the temperature of the reaction mixture, the extended primers will dissociate from the marker to form reaction products, excess primers will bind to the marker and to the reaction products and the process is repeated.

The reverse transcriptase (RT) PCR™ amplification procedure is a variant of PCR™ that permits amplification of mRNA templates. Thus, one may amplify the invasive mold DNA from a biological sample, by first isolating the mRNA and reverse transcribing this utilizing for example a RT-PCR kit (Invitrogen), according to the manufacturer's instructions.

(i) Quantitative PCR Methods

In some embodiments, the present invention relies on quantitative PCR™ to detect the presence of a infectious mold in a sample. These methods may be semi-quantitative or fully quantitative. Two approaches, competitive quantitative PCR™ (QPCR) and real-time quantitative PCR™, both estimate target gene concentration in a sample by comparison with standard curves constructed from amplifications of serial dilutions of standard DNA. However, they differ substantially in how these standard curves are generated. In competitive QPCR, an internal competitor DNA is added at a known concentration to both serially diluted standard samples and unknown (environmental) samples. After coamplification, ratios of the internal competitor and target PCR™ products are calculated for both standard dilutions and unknown samples, and a standard curve is constructed that plots competitor-target PCR™ product ratios against the initial target DNA concentration of the standard dilutions. Given equal amplification efficiency of competitor and target DNA, the concentration of the latter in environmental samples can be extrapolated from this standard curve.

In real-time QPCR, the accumulation of amplification product is measured continuously in both standard dilutions of target DNA and samples containing unknown amounts of target DNA. A standard curve is constructed by correlating initial template concentration in the standard samples with the number of PCR™ cycles (C_{T}) necessary to produce a specific threshold concentration of product. In the test samples, target PCR™ product accumulation is measured after the same C_{T}, which allows interpolation of target DNA concentration from the standard curve. Although real-time QPCR permits more rapid and facile measurement of target DNA during routine analyses, competitive QPCR remains an important alternative for target quantification in environmental samples. The coamplification of a known amount of competitor DNA with target DNA is an intuitive way to correct for sample-to-sample variation of amplification efficiency due to the presence of inhibitory substrates and large amounts of background DNA that are obviously absent from the standard dilutions.

Another type of QPCR is applied quantitatively PCR often termed “relative quantitative PCR,™ this method determines the relative concentrations of specific nucleic acids. In the context of the present invention, RT-PCR is performed on mRNA species isolated from patients. By determining that the concentration of a specific mRNA species varies, it is shown that the gene encoding the specific mRNA species is differentially expressed.

(a) Real Time PCR™

In some embodiments, the present invention utilizes quantitative real-time PCR-based methods to detect the presence of infective mold DNA in a biological sample. RNA or DNA, isolated from biological samples suspected of containing mold, may be amplified and simultaneously quantitated using the real-time PCR™ technique (Higuchi et al., 1993). This technique entails the use of a fluorogenic (or otherwise labeled) hybridization probes or dsDNA-specific fluorescent dyes to detect PCR™ product during amplification (real-time detection) without purification or separation by gel electrophoresis. The sensitivity of this method's probes allows measurement of the PCR™ product during the exponential phase of amplification before the critical reactants become limiting. This method does not require the separation of the PCR™ products.

The use of highly specific primers and fluorescent probe sequences are designed to yield target amplicons to unique regions of the invasive pathogenic mold genome. Thus, the inventors contemplate that rapid real-time PCR™-based diagnostic methods as described herein will allow for appropriate aggressive treatment regimens to be designed to treat the invasive mold.

Higuchi et al. (1993), constructed a system that detects PCR™ products in “real time” as they accumulate thereby allowing the analysis of PCR™ kinetics. This “real-time” system was comprised of an adapted thermal cycler to irradiate the samples with ultraviolet light, addition of the intercalator ethidium bromide in each amplification reaction, and detection of the resulting fluorescence with a computer-controlled cooled (CCD) camera. The amplification reaction produces increasing amounts of double-stranded DNA, which binds the ethidium bromide, resulting in an increase in fluorescence. By plotting the increase in fluorescence versus cycle number, the system produces amplification plots that provide a more complete picture of the PCR™ process than assaying product accumulation after a fixed number of cycles.

By determining the concentration of the amplified products of the target DNA or RNA in PCR™ reactions that have completed the same number of cycles and are in their linear ranges, it is possible to determine the relative concentrations of the specific target sequence in the original DNA mixture. This direct proportionality between the concentration of the PCR™ products and the relative mRNA or DNA abundances is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a RNA or DNA species can be determined by real-time PCR™ for a collection of RNA or DNA populations is that the concentrations of the amplified PCR™ products must be sampled when the PCR™ reactions are in the linear portion of their curves.

The second condition that must be met for a RT-PCR study to successfully determine the relative abundances of a particular mRNA species is that relative concentrations of the amplifiable cDNAs must be normalized to some independent standard. The goal of a real-time PCR experiment is to determine the abundance of a particular RNA or DNA species relative to the average abundance of all RNA or DNA species in the sample.

In recent developments to the real-time PCR™ methods, fluorescence is released in direct proportion to the accumulation of PCR™ product as the PCR™ proceeds in the TaqMan 5′ nuclease assay. Fluorescence production is continually monitored in each PCR™ reaction through the use of a CCD camera in each well of a multiwell format dish (typically 384 wells). The cycle number at which the fluorescence reaches a threshold value is precisely determined and used to calculate the amount of starting material by comparison to known standards. Typically, manufacturers' specifications claim a limit of detection of fifty copies of target molecule and a linear dynamic range of five orders of magnitude while several study reports indicate that even a single copy detection is possible and that ten copy detection is routine. The assay is also subject to high-throughput with approximately 1500 assays per day. Robotic fluid dispensing systems, multi-well plate fluorescent readers and the like can further increase the throughput of these assays.

Absolute versus Relative Quantitation. The quantity of starting material, either RNA or DNA, is determined by comparison with known standards. Comparing treated versus untreated cells or different cell types results in relative quantitation. Absolute quantitation requires the use of carefully quantitated external standards. Quantitation is typically performed by two methods, cyber green or TaqMan. The cyber green method tracks the accumulation of double stranded DNA and provides for a lower cost method of estimating gene copy number because only PCR™ primers are required. Higher accuracy is achieved by using the TaqMan FRET technology (fluorescence resonance energy transfer). These methods are known to the skilled artisan.

Fluorescent Detection Methods. Recent advances in fluorescence measurements using the light-emitting diode (LED) technology has led to the development of instrumentation coupled with PCR™ technology for real-time detection of specific PCR™ products. This real-time PCR™ technology offers several advantages over conventional PCR™ methods, such as a lower risk of PCR™ contamination, shorter turnaround time, and quantitative PCR™ analysis. Some of the most popular commercially available systems coupling PCR™ technology with real-time detection of PCR products include the ABI PRISM 7700 Sequence Detection System (PE Applied Biosystems, Foster City, Calif., USA), the LightCycler™ (Idaho Technologies, Idaho Fall, ID, USA; and Roche Diagnostics, Indianapolis, Ind., USA), and the Smart Cycler® (Cepheid, Sunnyvale, Calif., USA).

(ii) Theoretical Considerations

In PCR™, the number of molecules of the amplified target DNA increase by a factor approaching two with every cycle of the reaction until some reagent becomes limiting. Thereafter, the rate of amplification becomes increasingly diminished until there is no increase in the amplified target between cycles. If a graph is plotted in which the cycle number is on the X axis and the log of the concentration of the amplified target DNA is on the Y axis, a curved line of characteristic shape is formed by connecting the plotted points. Beginning with the first cycle, the slope of the line is positive and constant. This is said to be the linear portion of the curve. After a reagent becomes limiting, the slope of the line begins to decrease and eventually becomes zero. At this point the concentration of the amplified target DNA becomes asymptotic to some fixed value. This is said to be the plateau portion of the curve.

The concentration of the target DNA in the linear portion of the PCR™ amplification is directly proportional to the starting concentration of the target before the reaction began. By determining the concentration of the amplified products of the target DNA in PCR™ reactions that have completed the same number of cycles and are in their linear ranges, it is possible to determine the relative concentrations of the specific target sequence in the original DNA mixture. If the DNA mixtures are cDNAs synthesized from RNAs isolated from different tissues or cells, the relative abundances of the specific mRNA from which the target sequence was derived can be determined for the respective tissues or cells. This direct proportionality between the concentration of the PCR™ products and the relative mRNA abundances is only true in the linear range of the PCR™ reaction.

The final concentration of the target DNA in the plateau portion of the curve is determined by the availability of reagents in the reaction mix and is independent of the original concentration of target DNA. Therefore, the first condition that must be met before the relative abundances of a mRNA species can be determined by RT-PCR for a collection of RNA populations, is that the concentrations of the amplified PCR™ products must be sampled when the PCR™ reactions are in the linear portion of their curves.

The second condition that must be met for a quantitative RT-PCR experiment to successfully determine the relative abundances of a particular mRNA species is that relative concentrations of the amplifiable cDNAs must be normalized to some independent standard. The goal of an RT-PCR experiment is to determine the abundance of a particular mRNA species relative to the average abundance of all mRNA species in the sample. In the experiments described below, mRNAs for β-actin, asparagine synthetase and lipocortin II were used as external and internal standards to which the relative abundance of other mRNAs are compared.

Most protocols for competitive PCR™ utilize internal PCR™ standards that are approximately as abundant as the target. These strategies are effective if the products of the PCR amplifications are sampled during their linear phases. If the products are sampled when the reactions are approaching the plateau phase, then the less abundant product becomes relatively over represented. Comparisons of relative abundances made for many different RNA samples, such as is the case when examining RNA samples for differential expression, become distorted in such a way as to make differences in relative abundances of RNAs appear less than they actually are. This is not a significant problem if the internal standard is much more abundant than the target. If the internal standard is more abundant than the target, then direct linear comparisons can be made between RNA samples.

The above discussion describes theoretical considerations for an PCR™ or RT-PCR assay for clinically derived materials. The problems inherent in clinical samples are that they are of variable quantity (making normalization problematic), and that they are of variable quality (necessitating the co-amplification of a reliable internal control, preferably of larger size than the target). Both of these problems are overcome if the RT-PCR is performed as a relative quantitative RT-PCR with an internal standard in which the internal standard is an amplifiable cDNA fragment that is larger than the target cDNA fragment and in which the abundance of the mRNA encoding the internal standard is roughly 5-100 fold higher than the mRNA encoding the target. This assay measures relative abundance, not absolute abundance of the respective mRNA species.

Other studies may be performed using a more conventional relative quantitative RT-PCR assay with an external standard protocol. These assays sample the PCR™ products in the linear portion of their amplification curves. The number of PCR™ cycles that are optimal for sampling must be empirically determined for each target cDNA fragment. In addition, the reverse transcriptase products of each RNA population isolated from the various tissue samples must be carefully normalized for equal concentrations of amplifiable cDNAs. This consideration is very important since the assay measures absolute mRNA abundance. Absolute mRNA abundance can be used as a measure of differential gene expression only in normalized samples. While empirical determination of the linear range of the amplification curve and normalization of cDNA preparations are tedious and time consuming processes, the resulting RT-PCR assays can be superior to those derived from the relative quantitative RT-PCR assay with an internal standard.

One reason for this advantage is that without the internal standard/competitor, all of the reagents can be converted into a single PCR™ product in the linear range of the amplification curve, thus increasing the sensitivity of the assay. Another reason is that with only one PCR product, display of the product on an electrophoretic gel or another display method becomes less complex, has less background and is easier to interpret.

5. Other Amplification Procedures

A number of other template dependent processes are available to amplify the oligonucleotide sequences. For example, the ligase chain reaction (“LCR”), disclosed in European Application No. 320308, incorporated herein by reference in its entirety. U.S. Pat. No. 4,883,750 describes a method similar to LCR for binding probe pairs to a target sequence. A method based on PCR™ and oligonucleotide ligase assay (OLA), disclosed in U.S. Pat. No. 5,912,148, may also be used.

Alternative methods for amplification of target nucleic acid sequences that may be used in the practice of the present invention are disclosed in U.S. Pat. Nos. 5,843,650, 5,846,709, 5,846,783, 5,849,546, 5,849,497, 5,849,547, 5,858,652, 5,866,366, 5,916,776, 5,922,574, 5,928,905, 5,928,906, 5,932,451, 5,935,825, 5,939,291 and 5,942,391, GB Application No. 2,202,328, and in PCT Application No. PCT/US89/01025, each of which is incorporated herein by reference in its entirety.

Qbeta Replicase, described in PCT Application No. PCT/US87/00880, may also be used as an amplification method in the present invention. In this method, a replicative sequence of RNA that has a region complementary to that of a target is added to a sample in the presence of an RNA polymerase. The polymerase will copy the replicative sequence which may then be detected.

An isothermal amplification method, in which restriction endonucleases and ligases are used to achieve the amplification of target molecules that contain nucleotide 5′-[alpha-thio]-triphosphates in one strand of a restriction site may also be useful in the amplification of nucleic acids in the present invention (Walker et al., 1992). Strand Displacement Amplification (SDA), disclosed in U.S. Pat. No. 5,916,779, is another method of carrying out isothermal amplification of nucleic acids which involves multiple rounds of strand displacement and synthesis, i.e., nick translation.

Other nucleic acid amplification procedures include transcription-based amplification systems (TAS), including nucleic acid sequence based amplification (NASBA) and 3SR (Kwoh et al., 1989; Gingeras et al., PCT Application WO 88/10315, incorporated herein by reference in their entirety). European Application No. 329822 disclose a nucleic acid amplification process involving cyclically synthesizing single-stranded RNA (“ssRNA”), ssDNA, and double-stranded DNA (dsDNA), which may be used in accordance with the present invention.

PCT Application WO 89/06700 (incorporated herein by reference in its entirety) discloses a nucleic acid sequence amplification scheme based on the hybridization of a promoter region/primer sequence to a target single-stranded DNA (“ssDNA”) followed by transcription of many RNA copies of the sequence. This scheme is not cyclic, i.e., new templates are not produced from the resultant RNA transcripts. Other amplification methods include “race” and “one-sided PCR” (Frohman, 1994; Ohara et al., 1989).

### H. Separation Methods

It is normally desirable, at one stage or another, to separate the amplification products from reagents, such as the template or excess primers, or from other amplification products. For example, amplification products can be separated by agarose, agarose-acrylamide or polyacrylamide gel electrophoresis using standard methods. See Sambrook et al. (2001). When working with nucleic acids, denaturing PAGE is preferred.

Alternatively, chromatographic techniques may be employed to effect separation. There are many kinds of chromatography which may be used in the present invention such as, but not limited to, adsorption, partition, ion-exchange and molecular sieve, and many specialized techniques for using them including column, paper, thin-layer and gas chromatography (Freifelder, 1982). In yet another alternative, labeled cDNA products, such as biotin-labeled or antigen-labeled can be captured with beads bearing avidin or antibody, respectively.

Separated amplification products may be cut out and eluted from the gel for further manipulation. Using low melting point agarose gels, the separated band may be removed by heating the gel, followed by extraction of the nucleic acid.

These separation techniques can be adapted to function in the clinical setting, allowing the processing of large numbers of samples. However, new tools for the separation and detection of PCR products allow clinicians to view hundreds or thousands of samples at once. These techniques include FMAT (fluorometric microvolume assay technique), chemiluminescence, sequence detection systems (Applied Biosystems) and mass spectroscopy.

The following are a few examples of separation techniques readily applied to nucleic acids.

1. Microfluidic Techniques

Microfluidic techniques include separation on a platform such as microcapillaries, designed by ACLARA BioSciences Inc., or the LabChip™ “liquid integrated circuits” made by Caliper Technologies Inc. These microfluidic platforms require only nanoliter volumes of sample, in contrast to the microliter volumes required by other separation technologies. Miniaturizing some of the processes involved in genetic analysis has been achieved using microfluidic devices. For example, published PCT Application No. WO 94/05414, to Northrup and White, incorporated herein by reference, reports an integrated micro-PCR™ apparatus for collection and amplification of nucleic acids from a specimen. U.S. Pat. No. 5,304,487 to Wilding et al., and U.S. Pat. No. 5,296,375 to Kricka et al., discuss devices for collection and analysis of cell containing samples and are incorporated herein by reference. U.S. Pat. No. 5,856,174 describes an apparatus which combines the various processing and analytical operations involved in nucleic acid analysis and is incorporated herein by reference.

2. Capillary Electrophoresis

In some embodiments, it may be desirable to provide an additional, or alternative means for analyzing the amplified genes. In these embodiment, microcapillary arrays are contemplated to be used for the analysis.

Microcapillary array electrophoresis generally involves the use of a thin capillary or channel which may or may not be filled with a particular separation medium. Electrophoresis of a sample through the capillary provides a size based separation profile for the sample. The use of microcapillary electrophoresis in size separation of nucleic acids has been reported in, e.g., Woolley and Mathies (1994). Microcapillary array electrophoresis generally provides a rapid method for size-based sequencing, PCR™ product analysis and restriction fragment sizing. The high surface to volume ratio of these capillaries allows for the application of higher electric fields across the capillary without substantial thermal variation across the capillary, consequently allowing for more rapid separations. Furthermore, when combined with confocal imaging methods, these methods provide sensitivity in the range of attomoles, which is comparable to the sensitivity of radioactive sequencing methods. Microfabrication of microfluidic devices including microcapillary electrophoretic devices has been discussed in detail in, e.g., Jacobsen et al. (1994); Harrison et al. (1993); Manz et al. (1992); and U.S. Pat. No. 5,904,824. Typically, these methods comprise photolithographic etching of micron scale channels on a silica, silicon or other crystalline substrate or chip, and can be readily adapted for use in the present invention. In some embodiments, the capillary arrays may be fabricated from the same polymeric materials described for the fabrication of the body of the device, using the injection molding techniques described herein.

Tsuda et al. (1990), describes rectangular capillaries, an alternative to the cylindrical capillary glass tubes. Some advantages of these systems are their efficient heat dissipation due to the large height-to-width ratio and, hence, their high surface-to-volume ratio and their high detection sensitivity for optical on-column detection modes. These flat separation channels have the ability to perform two-dimensional separations, with one force being applied across the separation channel, and with the sample zones detected by the use of a multi-channel array detector.

In many capillary electrophoresis methods, the capillaries, e.g., fused silica capillaries or channels etched, machined or molded into planar substrates, are filled with an appropriate separation/sieving matrix. Typically, a variety of sieving matrices are known in the art may be used in the microcapillary arrays. Examples of such matrices include, e.g., hydroxyethyl cellulose, polyacrylamide, agarose and the like. Generally, the specific gel matrix, running buffers and running conditions are selected to maximize the separation characteristics of the particular application, e.g., the size of the nucleic acid fragments, the required resolution, and the presence of native or undenatured nucleic acid molecules. For example, running buffers may include denaturants, chaotropic agents such as urea or the like, to denature nucleic acids in the sample.

### I. Identification Methods

Amplification products must be visualized in order to confirm amplification of the target gene(s) sequences. One typical visualization method involves staining of a gel with for example, a flourescent dye, such as ethidium bromide or Vistra Green and visualization under UV light. Alternatively, if the amplification products are integrally labeled with radio- or fluorometrically-labeled nucleotides, the amplification products can then be exposed to x-ray film or visualized under the appropriate stimulating spectra, following separation.

In one embodiment, visualization is achieved indirectly, using a nucleic acid probe. Following separation of amplification products, a labeled, nucleic acid probe is brought into contact with the amplified gene(s) sequence. The probe preferably is conjugated to a chromophore but may be radiolabeled. In another embodiment, the probe is conjugated to a binding partner, such as an antibody or biotin, where the other member of the binding pair carries a detectable moiety. In other embodiments, the probe incorporates a fluorescent dye or label. In yet other embodiments, the probe has a mass label that can be used to detect the molecule amplified. Other embodiments also contemplate the use of Taqman and Molecular Beacon probes. In still other embodiments, solid-phase capture methods combined with a standard probe may be used as well.

The type of label incorporated in PCR™ products is dictated by the method used for analysis. When using capillary electrophoresis, microfluidic electrophoresis, HPLC, or LC separations, either incorporated or intercalated fluorescent dyes are used to label and detect the PCR™ products. Samples are detected dynamically, in that fluorescence is quantitated as a labeled species moves past the detector. If any electrophoretic method, HPLC, or LC is used for separation, products can be detected by absorption of UV light, a property inherent to DNA and therefore not requiring addition of a label. If polyacrylamide gel or slab gel electrophoresis is used, primers for the PCR™ can be labeled with a fluorophore, a chromophore or a radioisotope, or by associated enzymatic reaction. Enzymatic detection involves binding an enzyme to primer, e.g., via a biotin:avidin interaction, following separation of PCR™ products on a gel, then detection by chemical reaction, such as chemiluminescence generated with luminol. A fluorescent signal can be monitored dynamically. Detection with a radioisotope or enzymatic reaction requires an initial separation by gel electrophoresis, followed by transfer of DNA molecules to a solid support (blot) prior to analysis. If blots are made, they can be analyzed more than once by probing, stripping the blot, and then reprobing. If PCR™ products are separated using a mass spectrometer no label is required because nucleic acids are detected directly.

A number of the above separation platforms can be coupled to achieve separations based on two different properties. For example, some of the PCR™ primers can be coupled with a moiety that allows affinity capture, and some primers remain unmodified. Modifications can include a sugar (for binding to a lectin column), a hydrophobic group (for binding to a reverse-phase column), biotin (for binding to a streptavidin column), or an antigen (for binding to an antibody column). Samples are run through an affinity chromatography column. The flow-through fraction is collected, and the bound fraction eluted (by chemical cleavage, salt elution, etc.). Each sample is then further fractionated based on a property, such as mass, to identify individual components.

### J. Detection of Nucleic Acids

In accordance with the present invention, a nucleic amplification product will be detected and quantified. In certain applications, the detection may be performed by visual means. A typical visualization method involves staining of a gel with ethidium bromide and visualization of bands under UV light. Alternatively, if the amplification products are integrally labeled with radio- or fluorometrically-labeled nucleotides, the separated amplification products are subjected to radioactive scintigraphy of incorporated radiolabel or fluorescent detection, or using electrical and/or thermal impulse signals (Affymax technology; Bellus, 1994).

In one embodiment, following separation of amplification products, a labeled nucleic acid probe is brought into contact with the amplified marker sequence. The probe preferably is conjugated to a chromophore but may be radiolabeled. In another embodiment, the probe is conjugated to a binding partner, such as an antibody or biotin, or another binding partner carrying a detectable moiety.

In traditional methods, detection is by Southern blotting and hybridization with a labeled probe. The techniques involved in Southern blotting are well known to those of skill in the art (see Sambrook et al., 2001). One example of the foregoing is described in U.S. Pat. No. 5,279,721, incorporated by reference herein, which discloses an apparatus and method for the automated electrophoresis and transfer of nucleic acids. The apparatus permits electrophoresis and blotting without external manipulation of the gel and is ideally suited to carrying out methods according to the present invention.

Other methods of nucleic acid detection that may be used in the practice of the instant invention are disclosed in U.S. Pat. Nos. 5,840,873, 5,843,640, 5,843,651, 5,846,708, 5,846,717, 5,846,726, 5,846,729, 5,849,487, 5,853,990, 5,853,992, 5,853,993, 5,856,092, 5,861,244, 5,863,732, 5,863,753, 5,866,331, 5,905,024, 5,910,407, 5,912,124, 5,912,145, 5,919,630, 5,925,517, 5,928,862, 5,928,869, 5,929,227, 5,932,413 and 5,935,791, each of which is incorporated herein by reference.

1. Mass Spectroscopy

A recent innovation in nucleic acid detection is mass spectrometry. Mass spectrometry provides a means of “weighing” individual molecules by ionizing the molecules in vacuo and making them “fly” by volatilization. Under the influence of combinations of electric and magnetic fields, the ions follow trajectories depending on their individual mass (m) and charge (z). For low molecular weight molecules, mass spectrometry has been part of the routine physical-organic repertoire for analysis and characterization of organic molecules by the determination of the mass of the parent molecular ion. In addition, by arranging collisions of this parent molecular ion with other particles (e.g., argon atoms), the molecular ion is fragmented forming secondary ions by the so-called collision induced dissociation (CID). The fragmentation pattern/pathway very often allows the derivation of detailed structural information. Other applications of mass spectrometric methods in the known in the art can be found summarized in Methods in Enzymology, 1990.

Due to the apparent analytical advantages of mass spectrometry in providing high detection sensitivity, accuracy of mass measurements, detailed structural information by CID in conjunction with an MS/MS configuration and speed, as well as on-line data transfer to a computer, there has been considerable interest in the use of mass spectrometry for the structural analysis of nucleic acids. Reviews summarizing this field include Schram (1990); and Crain (1990). The biggest hurdle to applying mass spectrometry to nucleic acids is the difficulty of volatilizing these very polar biopolymers. Therefore, “sequencing” had been limited to low molecular weight synthetic oligonucleotides by determining the mass of the parent molecular ion and through this, confirming the already known sequence, or alternatively, confirming the known sequence through the generation of secondary ions (fragment ions) via CID in an MS/MS configuration utilizing, in particular, for the ionization and volatilization, the method of fast atomic bombardment (FAB mass spectrometry) or plasma desorption (PD mass spectrometry). As an example, the application of FAB to the analysis of protected dimeric blocks for chemical synthesis of oligodeoxynucleotides has been described (Koster et al., 1987).

Two ionization/desorption techniques are electrospray/ionspray (ES) and matrix-assisted laser desorption/ionization (MALDI). ES mass spectrometry was introduced by Fenn et al. (1989); WO 90/14148 and its applications are summarized in review articles (Smith et al., 1990; Ardrey, 1992). As a mass analyzer, a quadrupole is most frequently used. The determination of molecular weights in femtomole amounts of sample is very accurate due to the presence of multiple ion peaks which all could be used for the mass calculation.

MALDI mass spectrometry, in contrast, can be particularly attractive when a time-of-flight (TOF) configuration is used as a mass analyzer. The MALDI-TOF mass spectrometry has been introduced by Hillenkamp et al. (1990). Since, in most cases, no multiple molecular ion peaks are produced with this technique, the mass spectra, in principle, look simpler compared to ES mass spectrometry. DNA molecules up to a molecular weight of 410,000 daltons could be desorbed and volatilized (Williams et al., 1989). More recently, this the use of infra red lasers (IR) in this technique (as opposed to UV-lasers) has been shown to provide mass spectra of larger nucleic acids such as, synthetic DNA, restriction enzyme fragments of plasmid DNA, and RNA transcripts up to a size of 2180 nucleotides (Berkenkamp et al., 1998). Berkenkamp et al. (1998) also describe how DNA and RNA samples can be analyzed by limited sample purification using MALDI-TOF IR.

In Japanese Patent No. 59-131909, an instrument is described which detects nucleic acid fragments separated either by electrophoresis, liquid chromatography or high speed gel filtration. Mass spectrometric detection is achieved by incorporating into the nucleic acids atoms which normally do not occur in DNA such as S, Br, I or Ag, Au, Pt, Os, Hg.

2. Energy Transfer

Another emerging method for detecting nucleic acids involves energy transfer. Labeling hybridization oligonucleotide probes with fluorescent labels is a well known technique in the art and is a sensitive, non-radioactive method for facilitating detection of probe hybridization. More recently developed detection methods employ the process of fluorescence energy transfer (FET) rather than direct detection of fluorescence intensity for detection of probe hybridization. FET occurs between a donor fluorophore and an acceptor dye (which may or may not be a fluorophore) when the absorption spectrum of one (the acceptor) overlaps the emission spectrum of the other (the donor) and the two dyes are in close proximity. Dyes with these properties are referred to as donor/acceptor dye pairs or energy transfer dye pairs. The excited-state energy of the donor fluorophore is transferred by a resonance dipole-induced dipole interaction to the neighboring acceptor. This results in quenching of donor fluorescence. In some cases, if the acceptor is also a fluorophore, the intensity of its fluorescence may be enhanced. The efficiency of energy transfer is highly dependent on the distance between the donor and acceptor, and equations predicting these relationships have been developed (Forster, 1948). The distance between donor and acceptor dyes at which energy transfer efficiency is 50% is referred to as the Forster distance (R_{O}). Other mechanisms of fluorescence quenching are also known including, for example, charge transfer and collisional quenching.

Energy transfer and other mechanisms which rely on the interaction of two dyes in close proximity to produce quenching are an attractive means for detecting or identifying nucleotide sequences, as such assays may be conducted in homogeneous formats. Homogeneous assay formats are simpler than conventional probe hybridization assays which rely on detection of the fluorescence of a single fluorophore label, as heterogeneous assays generally require additional steps to separate hybridized label from free label. Several formats for FET hybridization assays are reviewed in Binninger, et al., (1992).

Homogeneous methods employing energy transfer or other mechanisms of fluorescence quenching for detection of nucleic acid amplification have also been described. Higuchi et al. (1999 and 1992) disclosed methods for detecting DNA amplification in real-time by monitoring increased fluorescence of ethidium bromide as it binds to double-stranded DNA. The sensitivity of this method is limited because binding of the ethidium bromide is not target specific and background amplification products are also detected. Lee et al. (1993) disclose a real-time detection method in which a doubly-labeled detector probe is cleaved in a target amplification-specific manner during PCR™. The detector probe is hybridized downstream of the amplification primer so that the 5-3 exonuclease activity of Taq polymerase digests the detector probe, separating two fluorescent dyes which form an energy transfer pair. Fluorescence intensity increases as the probe is cleaved. WO 96/21144 discloses continuous fluorometric assays in which enzyme-mediated cleavage of nucleic acids results in increased fluorescence. Fluorescence energy transfer is suggested for use in the methods, but only in the context of a method employing a single fluorescent label which is quenched by hybridization to the target.

Signal primers or detector probes which hybridize to the target sequence downstream of the hybridization site of the amplification primers have been described for use in detection of nucleic acid amplification (U.S. Pat. 5,547,861). The signal primer is extended by the polymerase in a manner similar to extension of the amplification primers. Extension of the amplification primer displaces the extension product of the signal primer in a target amplification-dependent manner, producing a double-stranded secondary amplification product which may be detected as an indication of target amplification. The secondary amplification products generated from signal primers may be detected by means of a variety of labels and reporter groups, restriction sites in the signal primer which are cleaved to produce fragments of a characteristic size, capture groups, and structural features such as triple helices and recognition sites for double-stranded DNA binding proteins.

Many donor/acceptor dye pairs known in the art and may be used in the present invention. These include, for example, fluorescein isothiocyanate (FITC)/tetramethylrhodamine isothiocyanate (TRITC), FITC/Texas Red.TM. (Molecular Probes), FITC/N-hydroxysuccinimidyl 1-pyrenebutyrate (PYB), FITC/eosin isothiocyanate (EITC), N-hydroxysuccinimidyl 1-pyrenesulfonate (PYS)/FITC, FITC/Rhodamine X, FITC/tetramethylrhodamine (TAMRA), and others. The selection of a particular donor/acceptor fluorophore pair is not critical. For energy transfer quenching mechanisms it is only necessary that the emission wavelengths of the donor fluorophore overlap the excitation wavelengths of the acceptor, i.e., there must be sufficient spectral overlap between the two dyes to allow efficient energy transfer, charge transfer or fluorescence quenching. P-(dimethyl aminophenylazo) benzoic acid (DABCYL) is a non-fluorescent acceptor dye which effectively quenches fluorescence from an adjacent fluorophore, e.g., fluorescein or 5-(2-aminoethyl) aminonaphthalene (EDANS). Any dye pair which produces fluorescence quenching in the detector nucleic acids of the invention are suitable for use in the methods of the invention, regardless of the mechanism by which quenching occurs. Terminal and internal labeling methods are both known in the art and maybe routinely used to link the donor and acceptor dyes at their respective sites in the detector nucleic acid.

3. Luminex

The Luminex technology allows the quantitation of nucleic acid products immobilized on color coded microspheres. The magnitude of the biomolecular reaction is measured using a second molecule called a reporter, and is done on individual microspheres as they flow through a detection chamber. The reporter molecule signals the extent of the reaction by attaching to the molecules on the microspheres. As both the microspheres and the reporter molecules are color coded, digital signal processing allows the translation of signals into real-time, quantitative data for each reaction.

### K. Kits

The invention also contemplates kits designed to detect the presence of invasive pathogenic molds that can cause IMI by the methods described herein. Thus, in some embodiments, the kits of the invention will comprise one or more components designed to be used in conducting real-time PCR™.

In a non-limiting example, the kits will comprise primers, probes, enzymes for reverse transcription, enzymes for amplification and additional agents such as buffers, nucleotides, water, and suitable positive and negative standards. The primers and probes may be lyophilized or may optionally be provided dissolved in a solvent containing such components as water, Tris, or other components known to those of ordinary skill in the art. The primers and probes may be provided labeled or with reagents that could be used to create labeled probe or primer. The buffers may include full strength or concentrated buffers, which may contain Tris, DMSO and/or other additives. The enzymes include Taq-polymerase or similar thermostable DNA polymerase used by those of skill in the art in conducting PCR, and reverse transcriptase, preferably in solution, such solution containing glycerol and/or water. Other reagents contemplated as useful include magnesium chloride and deoxynucleotides such as dATP, dCTP, dGTP and dTTP.

The kits may also comprise agents for DNA and/or RNA isolation and purification. The components of such kits will thus comprise one or more of these reagents in suitable container means.

The contents of such PCR™ kits and the foregoing compositions may be modified by those of ordinary skill in the art to achieve specific results and such kits and compositions are intended to be part of the present invention.

The components of the kits may be packaged either in aqueous media or in lyophilized form. The suitable container means of the kits will generally include at least one vial, test tube, flask, bottle, syringe or other container means, into which a component may be placed, and preferably, suitably aliquoted. Where there is more than one component in the kit, the kit also will generally contain a second, third or other additional container into which the additional components may be separately placed. However, various combinations of components may be comprised in a vial. The kits of the present invention also will typically include a means for containing the reagent containers in close confinement for commercial sale. Such containers may include injection or blow-molded plastic containers into which the desired vials are retained. Kits of the invention will also typically contain written instructions on the methods of using the kit.

### L. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Materials and Methods

Case Definition and Serum Samples. As described earlier, cases of IMI were defined according to the criteria established by EORTC and Mycoses Study Group (Ascioglu, et al., 2002). Depending on the degree of diagnostic certainty, the cases are defined as “definitive”, “probable”, “possible”, and “unlikely” IMI. “Definitive” (or documented) IMI represented a tissue diagnosis where branched septate hyphae, inflammation, and necrosis were seen microscopically and/or the fungus was successfully cultured from the tissue. Most of the patients in this group had pulmonary IMI and were typically neutropenic and/or immunosuppressed for an extended period of time. They exhibited prolonged pneumonia unresponsive to anti-bacterial therapy with nodular and/or cavitory lesions in the lung radiologically. Of the 13 patients with documented IMI in this study, tissue diagnoses were rendered in 12 patients by surgery or biopsy and one by autopsy. Patients with “probable IMI” typically were immunocompromised, had clinical and radiological features of IMI, and isolation of Aspergillus or other septate molds twice or more from respiratory specimens (such as sputa, bronchoalveolar lavages, and bronchial washings). Of the 28 patients in this category, 24 lacked tissue confirmation and 4 were autopsied after death later. Though pneumonia was seen in 3 of the autopsy cases, molds were not observed histologically or cultured microbiologically from the autopsy lung tissue. Thus, the IMI status of the four autopsy cases remained probable. Patients with “possible IMI” were those with atypical pulmonary radiology, pneumonia unresponsive to anti-bacterials, and isolation of mold once (rarely without) from the airway. Patients who did not meet the criteria for possible IMI represented “unlikely IMI.” Most patients with known or suspected (probable and possible) IMI had hematologic malignancies, in contrast to solid tumors in the majority of unlikely IMI patients.

Sera from these patients were collected retrospectively from clinical laboratories from October 1999 to September 2001 and stored at −20° C. until assay. A total of 559 serum samples from 106 patients were collected with single serum in 32 patients, 2 to 29 sera (mean 7) in 73 patients over a span of 1-267 days (median 22), and 7 sera in a single patient over 578 days. Most sera were drawn due to suspected episodes of infection. Thus, for patients with unlikely IMI, being low risk for IMI and other infections, fewer serial sera per patient were drawn (Table 1).

Fungal Strains and DNA. The mold DNA used as a quantitation standard was purified from A. fumigatus strain AF293 using standard cesium chloride density gradient. Candidal DNA was purified from clinical isolates using standard phenol-chloroform methods.

Serum DNA Extraction. Serum DNA was extracted using a silica-binding method (Boom et al., 1993), with modifications. Briefly, silica beads (Sigma-Aldrich, St. Louis, Mo.) were pretreated with diluted HCl, selected for particle size (1-10 μm) by serial settling in water, and stored at room temperature after autoclave. After serum (2 ml) was mixed with a lysis buffer (9 ml of 6 M guanidinium isothiocynate-Tris/HCl, pH 6) and incubated (37° C.×30 min), silica sludge (50 μl) was added and mixed thoroughly by vortex. The DNA-bound silica beads were pelleted through centrifugation (6000 rpm×10 min), washed once more with the lysis buffer, twice with ethanol, and finally with acetone, then dried. The beads were resuspended in 50 μL of Tris-EDTA buffer (10 mM, pH 7) and the bound DNA was eluted off the beads through filtration (0.45 μm filter, Alltech, Deerfield, Ill.) and centrifugation (12,000 rpm×1 min). Purified DNA was measured at 260 nm and stored at −20° C.

Design of Primers and Probe. The sequences of primers and probe were designed based on those of A. fumigatus. After evaluation of several candidate genes, the 5.8S ribosomal RNA gene was chosen for its conservation among various Aspergillus species (A. fumigatus, A. flavus, A. terreus, A. niger and others). The sequences were selected to avoid significant homology to and thus amplification of yeast and human sequences. Additional guidelines for real-time PCR design of primers and probe were also followed (Applied Biosystems, Foster City, Calif.). In pilot experiments, a number of primers in various combinations were tested for sensitivity and specificity. The optimal primers were found to be 5′-TTGGTTCCGGCATCGA (SEQ ID. NO:2), and 5′-GCAGCAATGACGCTCGG (SEQ ID. NO:3), corresponding to positions 235 to 376 (142 base pairs) of GenBank accession AF138288 (SEQ ID. NO:1). The probe was 5′-6-FAM-TGAAGAACGCAGCGAAATGCGATAA-TAMRA, (SEQ ID. NO:4), where FAM and TAMRA were fluorescent dyes 6-carboxyfluorescein and 6-carboxy-N,N,N′,N′-tetramethylrhodamine, respectively. These sequences (total 58 nucleotides) match significantly to those of Fusarium (53 of 58, F. solani, AF178419 (SEQ ID. NO:5)) and Scedosporium (54 of 58, S. prolificans, AF022485 (SEQ ID. NO:6)) and allowed amplification of these molds as well. The sequence homology to other rare molds, however, was not assessed nor amplification tested.

Amplification. Real-time PCR™ was performed on Prism 7700 Sequence Detection System (Applied Biosystems, Foster City, Calif.). Purified serum DNA (total up to 1 μg in 20 μl) was added to the reaction mix (final 25 μl) containing 1 μM each of the primers, 200 nM fluorescent-labeled probe, 400 μM dNTP, 5 mM MgCl₂, and 1.5U TaqGold polymerase. Following activation of polymerase (95° C.×10 min), 40 thermocycles were run with denaturation (95° C.×20 s), annealing (63° C.×20 s) and extension (72° C.×30 s). The last extension was 2 min. To prevent contamination, universal precautions and one-way flow of DNA extraction and amplification were exercised. To avoid potential subjectivity, the IMI status of each patient was unknown during the PCR™ assay.

Data Analysis. Where appropriate, statistical analysis was performed by using either chi-square or the Fisher's exact test.

### Example 2

### Results

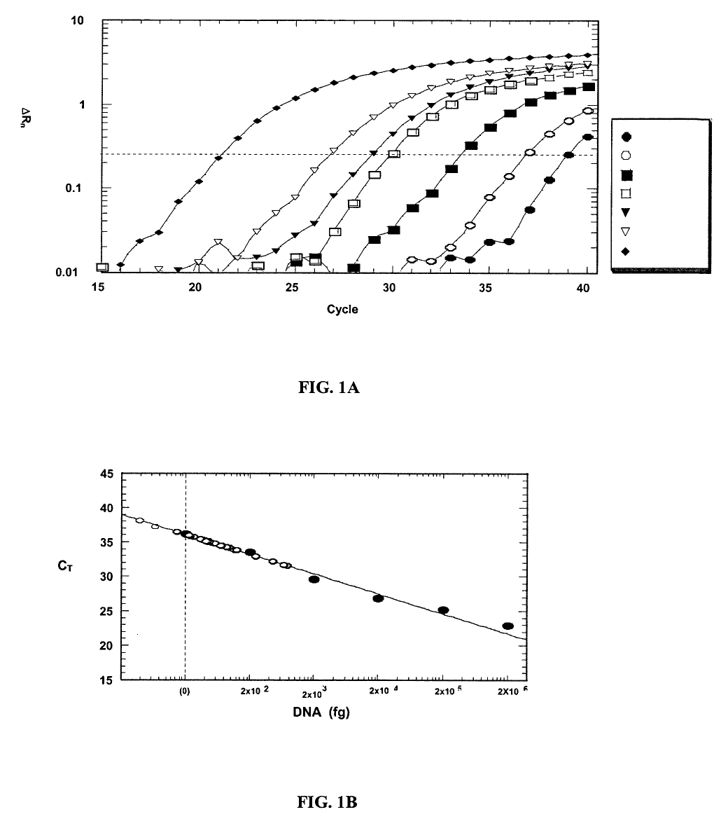

Specificity and Detection Range. The specificity and detection range of the real-time PCR were assessed with purified Aspergillus, human, and candidal DNA. Neither human nor candidal DNA was amplified (data not shown). With normal human DNA as a background, purified Aspergillus DNA from 20 ng to 200 fg (5-log range) was detected at various amplification cycles (FIG. 1A). A logarithmic plot of the DNA quantity correlated linearly with the number of cycles (FIG. 1B), thus providing a basis for quantitative analysis of patient specimens.

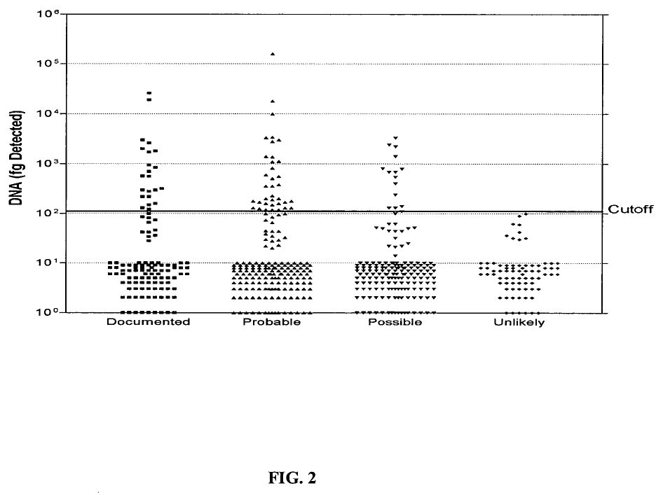

Test of Sera. A total of 559 serum samples from 106 patients were tested with this real-time PCR assay and the results are shown in FIG. 2. All 76 sera from 35 patients with no evidence of IMI showed undetectable (less than negative control, <10 fg) or very low (10-100 fg) DNA levels so that a cutoff of 110 fg could be drawn confidently. This cutoff approximated 3 mold genomes for sensitivity and also enabled excellent specificity of the assay (100%). It allowed objective assessment of a positive test and categorization as weak (110-999 fg), moderate (1,000 to 10,000 fg), or strong (>10,000 fg) positivity (FIG. 2). At this cutoff, sera from patients with documented and suspected (probable and possible) IMI showed varying percentages of positivity that correlated with the diagnostic certainty of IMI, as shown in FIG. 2.

The test results are also summarized in Table 1. A patient was considered test-positive if one or more of his/her sera was positive. It was realized that this practical definition might cause potential bias towards more positive patients, due to multiple sera per patient, in the documented and suspected IMI groups as compared to fewer such serial sera in the unlikely IMI group. To minimize this, comparison was also made by the number of sera per group. Sera from documented and probable IMI had the same positive rate of 19%, which probably reflected similar disease status of the two groups. Many patients with probable IMI were short of tissue confirmation because of deeper lesions in the lung and risk factors unsuitable for invasive procedures. Possible IMI sera had a positive rate of 9% (15 of 176), significantly lower than 19% (58 of 307, combined documented and probable IMI) (χ2=9.41, p<0.01). The overall positive rate for sera of these groups was 15% (73 of 483). When analyzed by the number of patients, the positive rate was 59% (42 of 71) for these three groups. A gradient was similarly seen with 40% (12 of 30 patients) for possible IMI, 68% (19 of 28) for probable IMI, and 85% (11 of 13) for documented IMI. Those patients in each group had mean 6 to 9 sera with median sampling time span of 10-16 days, suggesting good comparability.

TABLE 1
Serum real-time PCR ™ for patients with varying probability of IMI

| IMI | No. | No. | % | No. | No. | % | Median Sample |
|---|---|---|---|---|---|---|---|
| Category | Patients | Positive | Positive | Sera | Positive | Positive | span ^{a}(day) |
| Documented | 13 | 11 | 85 | 120 | 23 | 19 | 13 |
| Probable | 28 | 19 | 68 | 187 | 35 | 19 | 36 |
| Possible | 30 | 12 | 40 | 176 | 15 | 9 | 10 |
| Subtotal | 71 | 42 | 59 | 483 | 73 | 15 | 16 |
| Unlikely | 35 | 0 | 0 | 76 | 0 | 0 | 1 |
| Total | 106 | 42 | 40 | 559 | 73 | 13 | 7 |

^{a}See Materials and Methods for detail.

An attempt was also made to analyze all the 73 positive sera and estimate the associated mold DNA burden in those patients (Table 2). At the assay cutoff of 110 fg, using dilution factor of 2.5 and total serum volume of 3,500 ml (70% of 5,000 ml blood volume), total body circulating mold DNA was estimated to be 3×10⁴ genomes. At the geometric mean positive level of 580 fg, the burden was 5.3 ng or 1.6×10⁵ genomes. The heaviest burden corresponded to 4.2×10⁷ genomes or 42 μg in a patient with end stage IA.

TABLE 2
Estimated Mold DNA Burden From Positive Sera (n = 73)

| | Geometric | Range |
|---|---|---|
| | mean | (n = 73) |
| DNA detected (fg) (copies of | 580 (17) | 110-160,000 |
| genome) | | (3-4,800) |
| Circulating DNA concentration | 1,500 | 280-400,000 |
| (fg/ml)^{a} | | |
| Total circulating DNA (ng) (genomes | 5.3 (160) | 1.0-1,400 |
| × 10³)^{b} | | (30-42,000) |

^{a}Dilution factor = 2.5
^{b}Estimated circulating serum volume of 3500 ml; each ng = ˜30,000 copies of mold (Aspergillus) genome; 1 ng = 10⁶ fg

Analysis of Mold Isolates. From 1998 to 2000, 406 patients at the MD Anderson Cancer Center (Houston, Tex.) had Aspergillus isolated from the respiratory tract and/or tissue. Among these patients, the Aspergillus distributions were A. fumigatus in 97 patients (24% of 406), A. flavus in 93 (23%), A. terreus in 87 (21%), A. niger in 75 (18%), other species in 10 (2%), and multiple species in 44 (11%). During the same period, Fusarium was isolated from 73 patients and Scedosporium from 13 patients. In this study, patients from the IMI groups represented a portion during this period and their PCR results were analyzed according to the mold types (Table 3). Consistent with the 3-year data and literature findings, A. fumigatus and A. flavus were the most common species and 52% of these patients (13 of 25) were shown to be positive by PCR. Notably, A. terreus was isolated from 14 patients (9 as single isolate and 5 more as second species), 11 of whom had probable IMI and 3 possible IMI. Nine of the 14 patients (64%) were also PCR-positive. Therefore, A. terreus, being isolated nearly as common as A. fumigatus and A. flavus, represented another major species associated with IA. In contrast, only 4 patients (3 possible and 1 probable IA) had A. niger isolated alone, and none was PCR-positive. This rate (0/4) was significantly lower than that of all other Aspergillus spp. combined (58%, 32 of 55) (p=0.039, Fisher's exact test). This finding is consistent with the literature that A. niger is infrequently associated with IA (Paterson and Singh, 1999; Latge, 1999).

Some of the IMI patients were afflicted by Fusarium, a mold that is difficult to be distinguished from Aspergillus in tissue phase by morphology. Fusarium can be cultured from the bloodstream and, unsurprisingly, all 6 patients with fusariosis showed detectable DNA in the serum (Table 3). One patient with probable scedosporiosis also tested positive (Table 3). Three of five patients who had possible IMI based on strong clinical suspicion without a mold being cultured also tested positive. This result is not surprising in view of previous studies by the inventors which demonstrated frequent culture failure for molds (Tarrand et al., 2000).

TABLE 3
Mold isolates and serum PCR from patients
with documented and suspected IMI

| Mold | No. of patients | PCR (+) | % |
|---|---|---|---|
| Aspergillus spp. | | | |
| A. fumigatus | 14 | 8 | 57 |
| A.flavus | 11 | 5 | 45 |
| A. terreus | 9 | 7 | 78 |
| A. niger | 4 | 0 | 0 |
| Other species | 10 | 7 | 70 |
| Two or more species | 11 | 5 | 45 |
| Subtotal | 59 | 32 | 54 |
| Fusarium | 6 | 6 | 100 |
| Scedosporium | 1 | 1 | 100 |
| Clinical diagnosis only^{a} | 5 | 3 | 60 |
| Total | 71 | 42 | 59 |

^{a}These five patients all had possible IMI, based on strong clinical suspicion.

Thus, the present invention provides real-time PCR-based methods for detecting septate mold DNA specifically, sensitively, and quantitatively in the sera of patients with IMI. The specificity was found to be excellent, being test-negative in all patients with no evidence of IMI. The sensitivity was also good, being 85% among patients with documented IMI that were diagnosed by a combined assessment of histology, microbiology, radiology and clinical manifestations. This assay is convenient and minimally invasive, thus making it a potential alternative to other invasive diagnostic procedures, such as biopsy and open surgery. In many patients with hematological malignancies, these procedures are too risky to be performed due to bleeding, additional infections, etc. Thus, this assay eliminates the possibility of cross-contamination by PCR™ products since simultaneous probing and detection omits the post-PCR manipulation that is needed in conventional PCR™ assays and prone to contamination. Like any other diagnostic tests, multiple sampling may be necessary. Since most positive patients had IA (at least 76%, 32 of 42 patients, Table 3), a positive test may be presumptive of IA, until the identification of the mold by culture.

The outcome of IMI and the level of circulating mold DNA may also be influenced by several factors, such as the use of anti-fungal therapeutics, remission of underlying disease, and resolution or improvement of active IMI or its risk factors. Many of the patients in this group were also treated with experimental anti-fungals in addition to amphotericin and itraconazole.

A. terreus was also found to be a frequent cause of IA in this study in addition to A. fumigatus and A. flavus. Patients with suspected A. terreus infection alone showed the highest positive rate for circulating DNA (7 of 9, Table 3), raising the possibility that fungemia may be more common with this species. Indeed, the inventors previous study also reported true fungemia caused by A. terreus, but not by other Aspergillus species (Kontoyiannis et al., 2000). Thus, it is speculated that A. terreus may be more angio-invasive and further study is needed to gain insight into this aspect. The overall recovery of Aspergillus from blood culture is very low, which precludes assessment of circulatory burden imposed by this organism. Possible explanations include fragility or low numbers of viable organisms in the bloodstream, transient shedding into the circulation, inhibitory factors in the blood, failure of current culture techniques, or combinations of these. Nonetheless, the present quantitative PCR allowed estimation of the mold DNA in the circulation (free, cell-bound, and fragmented forms from viable or dead organisms). At the lowest detectable level, circulative mold burden was ˜3×10⁴ genomes. The geometric mean was 5-fold higher (1.6×10⁵ genomes) and the heaviest burden was 4.2×10⁷ genomes (Table 2). In a recent mouse model study of disseminated aspergillosis, quantitative PCR™ has also been used to measure the burden of A. fumigatus, which correlated with disease progression and therapeutic efficacy (Bowman, et al., 2001). The inventors are currently investigating these issues in patients.

Galactomannan is a complex polysaccharide antigen and major cell wall constituent of Aspergillus (Latge, 1999). Detection of galactomannan in the circulation by immunoassay has also become a useful diagnostic tool for IA. A recent prospective study revealed a sensitivity of 90% and a specificity of 98% (Maertens et al., 2001). These results are similar to the findings by real-time PCR™. However, false positive reactions are relatively frequent in galactomannan assays, particularly in the format of enzyme-linked immunosorbent assay, being 14% in the above-mentioned study and 8% in another (Stynen et al., 1995). Admittedly, lack of specificity can also be problematic with PCR™ due to the exponential amplification of the target DNA. For instance, non-specificity was a likely factor in a nested PCR™ study that showed unusually high positive rates of sera, 11% for patients with unlikely and indeterminate IA and 61% for patients with possible to proven IA (Williamson et al., 2000). In the present invention, this potential pitfall was avoided or minimized through careful design of primers and probe and selection of cutoff value. As such, the overall serum positive rate of 15% in the IMI groups (Table 1) is more realistic. The present methods based on real-time PCR, being more specific and comparably sensitive, would correct the false positive results by the galactomannan assay if both assays were used in suspected IA patients.

Efficient DNA extraction is crucial for any blood-based PCR™ assay, particularly to detect microbial DNA. The silica-binding method used in this study was able to extract extremely low levels (picogram) of fungal DNA along with microgram levels (10⁶-fold more) of human DNA in a serum sample. It was superior to the phenol-chloroform method and some commercial DNA extraction kits (data not shown). A previous study (Fahle, et al., 2000), also reported that the silica-binding method was the best among six methods in extracting cytomegalovirus DNA from serum and cerebrospinal fluid. The inventors chose serum to extract mold DNA because it appears to be a better source than plasma or whole blood for the extraction of fungal or bacterial DNA (Yamakami et al., 1996; Zerva et al., 2001). In addition, serum typically has much less human DNA (1-5 μg/ml) than whole blood (30-50 μg/ml), which might favor the extraction of minute quantities of exogenous microbial DNA.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### REFERENCES

The following references, to the extent that they provide exemplary procedural or other details supplementary to those set forth herein, are specifically incorporated herein by reference.
U.S. Pat. No. 4,659,774
U.S. Pat. No. 4,683,202
U.S. Pat. No. 4,800,159
U.S. Pat. No. 4,816,571
U.S. Pat. No. 4,883,750
U.S. Pat. No. 4,959,463
U.S. Pat. No. 5,141,813
U.S. Pat. No. 5,264,566
U.S. Pat. No. 5,279,721
U.S. Pat. No. 5,296,375
U.S. Pat. No. 5,304,487
U.S. Pat. No. 5,428,148
U.S. Pat. No. 5,547,861
U.S. Pat. No. 5,547,861
U.S. Pat. No. 5,554,744
U.S. Pat. No. 5,574,146
U.S. Pat. No. 5,602,244
U.S. Pat. No. 5,840,873
U.S. Pat. No. 5,843,640
U.S. Pat. No. 5,843,650
U.S. Pat. No. 5,843,651
U.S. Pat. No. 5,843,663
U.S. Pat. No. 5,846,708
U.S. Pat. No. 5,846,709
U.S. Pat. No. 5,846,717
U.S. Pat. No. 5,846,726
U.S. Pat. No. 5,846,729
U.S. Pat. No. 5,846,783
U.S. Pat. No. 5,849,481
U.S. Pat. No. 5,849,486
U.S. Pat. No. 5,849,487
U.S. Pat. No. 5,849,497
U.S. Pat. No. 5,849,546
U.S. Pat. No. 5,849,547
U.S. Pat. No. 5,851,772
U.S. Pat. No. 5,853,990
U.S. Pat. No. 5,853,992
U.S. Pat. No. 5,853,993
U.S. Pat. No. 5,856,092
U.S. Pat. No. 5,856,174
U.S. Pat. No. 5,858,652
U.S. Pat. No. 5,861,244
U.S. Pat. No. 5,863,732
U.S. Pat. No. 5,863,753
U.S. Pat. No. 5,866,331
U.S. Pat. No. 5,866,366
U.S. Pat. No. 5,900,481
U.S. Pat. No. 5,904,824
U.S. Pat. No. 5,905,024
U.S. Pat. No. 5,910,407
U.S. Pat. No. 5,912,124
U.S. Pat. No. 5,912,145
U.S. Pat. No. 5,912,148
U.S. Pat. No. 5,916,776
U.S. Pat. No. 5,916,779
U.S. Pat. No. 5,919,626
U.S. Pat. No. 5,919,630
U.S. Pat. No. 5,922,574
U.S. Pat. No. 5,925,517
U.S. Pat. No. 5,928,862
U.S. Pat. No. 5,928,869
U.S. Pat. No. 5,928,905
U.S. Pat. No. 5,928,906
U.S. Pat. No. 5,929,227
U.S. Pat. No. 5,932,413
U.S. Pat. No. 5,932,451
U.S. Pat. No. 5,935,791
U.S. Pat. No. 5,935,825
U.S. Pat. No. 5,939,291
U.S. Pat. No. 5,942,391
Ardrey, Electrospray Mass Spectrometry, Spectroscopy Europe, 4:10-18, 1992.
Ascioglu et al., Clin. Infect. Dis., 34:7-14, 2000.
Baddley et al., Clin. Infect. Dis., 32(9):1319-24, 2001.
Bellus, J. Macromol. Sci. Pure Appl. Chem., A31(1): 1355-1376, 1994.
Berkenkamp et al., Science, 281(5374):260-2, 1998.
Boom et al., J. Clin. Microbiol., 28:495-503, 1990.
Bowman et al., Antimicrob. Agents Chemother., 45:3474-81, 2001.
Binninger et al., Diagn Mol Pathol. December;1(4):239-45, 1992.
Brakhage and Langfelder, Annu. Rev. Microbiol., 56:433-455, 2000.
Bretagne et al., Clin. Infect. Dis., 26:1407-1412, 1998.
Chomczynski and Sacchi, Anal Biochem., April;162(1):156-9, 1987.
Crain, “In: Mass Spectrometry Reviews, 9:505-554, 1990.
Effenhauser et al Anal. Chem., 66:2949-2953, 1994.
Effenhauser et al. Anal. Chem., 65:2637-2642, 1993.
Einsele et al., J. Clin. Microbiol., 35:1353-60, 1997.
European Application No. 320308
European Application No. 329 822
Fahle et al., J Clin. Microbiol., 38:3860-3, 2000.
Fenn et al., J. Phys. Chem. 88:4451-4459, 1984
Fodor et al., Nature, 364:555-556, 1993.
Forster Ann. Phys. 2:55-75, 1948.
Freifelder, Physical Biochemistry Applications to Biochemistry and Molecular Biology, 2nd ed. Wm. Freeman and Co., New York, N.Y., 1982.
Frohman, In: PCR Protocols: A Guide To Methods And Applications, Academic Press, N.Y., 1990.
GB Application No. 2 202 328
Hacia et al., In: Nature Genetics 14, 1996.
Harrison et al., Science, 261:895-897, 1993.
Hebart et al., J. Infect. Dis., 181:1713-9, 2000.
Higuchi et al., Biotechnology, 11(9):1026-30, 1993.
Higuchi et al., Biotechnology 10:413-417 1992.
Hillenkamp et al., In: Biological Mass Spectrometry, Burlingame and McCloskey (ed), Elsevier Science Publishers, Amsterdam, pp. 49-60, 1990.
Innis et al., PCR Protocols, Academic Press, Inc., San Diego Calif., 1990.
Itakura and Riggs, Science, 209:1401-1405, 1980.
Jacobson et al., Anal. Chem., 66:1107-1113, 1994.
Japanese Pat. No. 59-131909
Japanese Patent No. 59-131909
Kontoyiannis et al., Clin. Infect. Dis., 31:188-189, 2000.
Koster et al., In: Biomedical Environmental Mass Spectrometry, 14:111-116, 1987.
Kwoh et al., Proc. Nat. Acad. Sci. USA, 86: 1173, 1989.
Latge Clin. Microbiol. Rev., 12:310-50, 1999.
Lee et al., Nuc. Acids Res., 21:3761-3766, 1993.
Maertens et al., Blood, 97:1604-10, 2001.
Maniatis et al., Molecular Cloning, A Laboratory Manual, Cold Spring Harbor Press, Cold Spring Harbor, N.Y., 1988.
Manz et al., J. Chromatogr., 593:253-258, 1992.
Methods in Enzymology, In: Mass Spectrometry, McCloskey (ed.), Vol. 93, Academic Press, New York, 1990.
Nonisotopic DNA Probe Techniques, Academic Press, Inc., 311-352, 1992.
Ohara et al., Proc. Natl. Acad. Sci. USA, 86:5673-5677, 1989.
Oliveria et al., Braz. J. Med. Biol. Res., 35(7):789-98, 200, 2002.
Paterson and Singh, Medicine, 78:123-128, 1999.
PCT Application No. PCT/US87/00880
PCT Application No. PCT/US89/01025
PCT Application WO 88/10315
PCT Application WO 89/06700
PCT Application WO 94/05414
Pease et al., Proc. Natl. Acad. Sci. USA, 91:5022-5026, 1994.
Sambrook, Molecular Cloning: A Laboratory Manual, 3rd Ed., Cold Spring Harbor Press, Cold Spring Harbor, N.Y., 2001.
Schram, In: Biomedical Applications of Mass Spectrometry, 34:203-287, 1990.
Shoemaker et al.,
Nature Genetics, 14:450-456, 1996.
Smith et al., Anal. Chem., 62, 882-89, 1990.
Stynen et al., J. Clin. Microbiol., 33:497-500, 1995.
Tarrand et al., 38^{th} Annual Meeting of the Infectious Diseases Society of America (IDSA), abstract #276, 2000.
Tsuda et al., Anal. Chem., 62:2149-2152, 1990.
Van Burik et al., J. Clin. Microbiol., 36:1169-75, 1998.
Vogeser et al., Eur. J. Clin. Microbiol. Infect. Dis. 16:1-6, 1997.
Walker et al., Proc. Natl. Acad. Sci. USA, 89:392-396 1992.
Williams et al., Science, 246:1585-87, 1989.
Williamson et al., Br. J. Haematol., 108:132-9, 2000.
WO 90/07641
WO 90/14148
WO 96/21144
Woolley and Mathies, Proc. Natl. Acad. Sci. USA, 91:11348-52 1994.
Yamakami et al., J. Clin. Microbiol., 34:2464-8, 1996.
Yamakami et al., J. Clin Microbiol 1996;34:2464-8.
Zerva et al., J. Clin. Microbiol., 39:1661-4, 2001.

1. A method for detecting the presence of an invasive mold in a subject comprising identifying 5.8S ribosomal RNA of an invasive mold, or a DNA encoding said RNA in a sample obtained from the subject.

2. The method of claim 1, comprising mixing the sample with primers that hybridize to the RNA.

3. The method of claim 2, further comprising amplifying the sample comprising the RNA.

4. The method of claim 3, comprising determining the presence or absence of an amplification product in the sample wherein the presence of an amplification product is indicative of the presence of an invasive mold.

5. The method of claim 1, wherein the sample is a nucleic-acid containing sample

6. The method of claim 5, wherein the nucleic-acid containing sample is a DNA sample.

7. The method of claim 5, wherein the nucleic-acid containing sample is a RNA sample.

8. The method of claim 4, further comprising quantitating the amplification product whereby the amount of invasive mold nucleic acid is quantitated.

9. The method of claim 8, wherein said quantitating comprises:
a) mixing a first probe capable of hybridizing to a nucleic acid sequence of said invasive mold in an amplification reaction;
b) mixing a second probe capable of hybridizing to a standard nucleic acid that is amplified to a pre-determined quantity in the amplification reaction of step (a); and
c) comparing the amount of the first probe to the amount of the second probe.

10. The method of claim 9, wherein the mixing is in the linear phase of the amplification.

11. The method of claim 9, wherein the first probe comprises nucleic acids that hybridize to the nucleic acid sequence of SEQ ID NO:1, SEQ ID NO:5 or SEQ ID NO:6 or fragments thereof.

12. The method of claim 11, wherein the probe comprises the sequence 5′-TGAAGAACGCAGCGAAATGCGATAA-3′ (SEQ ID. NO:4).

13. The method of claim 9, wherein said probe is labeled.

14. The method of claim 13, wherein the label is a fluorescent label.

15. The method of claim 14, wherein said fluorescent label is 6-carboxyfluorescein (FAM), 6-carboxy-N,N,N′,N′-tetramethylrhodamine (TAMRA), Alexa 350, Alexa 430, AMCA, BODIPY 630/650, BODIPY 650/665, BODIPY-FL, BODIPY-R6G, BODIPY-TMR, BODIPY-TRX, Cascade Blue, Cy3, Cy5,6-FAM, Fluorescein, HEX, 6-JOE, Oregon Green 488, Oregon Green 500, Oregon Green 514, Pacific Blue, REG, Rhodamine Green, Rhodamine Red, ROX, TAMRA, TET, or Texas Red.

16. The method of claim 15, wherein the probe comprises the sequence 5′-6-FAM-TGAAGAACGCAGCGAAATGCGATAA-TAMRA-3′ (SEQ ID NO:4).

17. The method of claim 3, wherein the amplifying is preceded by a reverse transcription reaction.

18. The method of claim 1, wherein the invasive mold belongs to Aspergillus species, Fusarium species, or Scedosporium species.

19. The method of claim 18, wherein said mold is of the Aspergillus species.

20. The method of claim 19, wherein said mold is Aspergillus fumigatus, Aspergillus flavus, Aspergillus terreus, Aspergillus vesicularis, Aspergillus nidulans, or Aspergillus niger.

21. The method of claim 18, wherein said invasive mold is of the Fusarium species.

22. The method of claim 21, wherein said mold is Fusarium solani.

23. The method of claim 18, wherein said invasive mold is of the Scedosporium species.

24. The method of claim 23, wherein said invasive mold is Scedosporium prolificans.

25. The method of claim 1, where said sample comprises serum, blood, plasma, cells, tissues, aspirates, biopsies, fine needle aspirates, skin biopsies, lymph fluid or urine.

26. The method of claim 1, where said sample comprises serum.

27. The method of claim 2, wherein said primers are comprised of nucleic acids that hybridize to the nucleic acid sequence comprised in SEQ ID NO: 1 or fragments thereof.

28. The method of claim 27, wherein said primers comprise the nucleic acid sequence TTGGTTCCGGCATCGA (SEQ ID. NO:2) or GCAGCAATGACGCTCGG (SEQ ID. NO:3).

29. The method of claim 2, wherein said primers are comprised of nucleic acids that hybridize to the nucleic acid sequence comprised in SEQ ID NO: 5 or fragments thereof.

30. The method of claim 2, wherein said primers are comprised of nucleic acids that hybridize to the nucleic acid sequence comprised in SEQ ID NO: 6 or fragments thereof.

31. The method of claim 3, wherein said amplifying is by polymerase chain reaction (PCR™).

32. The method of claim 4, wherein said determining is in real time.

33. The method of claim 1, wherein the detecting is in a detection range of 200 fg to 20 ng of DNA.

34. The method of claim 1, further comprising obtaining said sample from the subject.

35. The method of claim 1, further comprising isolating nucleic acids from said sample.

36. A kit for detecting an invasive mold in a biological sample comprising:
a) primers that hybridize to 5.8S ribosomal RNA of an invasive mold, or DNA encoding said RNA; and
b) reagents for an amplification reaction comprising a heat-stable DNA polymerase enzyme, buffers, water, magnesium chloride, and deoxynucleotides;
each enclosed in a suitable container means.

37. The kit of claim 36, wherein the primers comprise nucleic acids to the nucleic acid sequence of SEQ ID NO:1, SEQ ID NO:5 or SEQ ID NO:6 or fragments thereof.

38. The kit of claim 36, wherein the primers comprise the nucleic acid sequence TTGGTTCCGGCATCGA (SEQ ID. NO:2) or GCAGCAATGACGCTCGG (SEQ ID. NO:3).

39. The kit of claim 36, further comprising one or more probe that hybridize to ribosomal RNA of the invasive mold or fragments thereof.

40. The kit of claim 39, wherein the ribosomal RNA comprises one or more probe that hybridize to the 5.8S ribosomal RNA of the invasive mold or fragments thereof.

41. The kit of claim 39, wherein said probes comprise nucleic acids that hybridize to the nucleic acid sequence of SEQ ID NO:1, SEQ ID NO:5 or SEQ ID NO:6 or fragments thereof.

42. The kit of claim 41, wherein the probe comprises the sequence 5′-TGAAGAACGCAGCGAAATGCGATAA-3′ (SEQ ID. NO:4).

43. The kit of claim 39, wherein said probe is labeled.

44. The kit of claim 36, further comprising reagents to isolate nucleic acids from the sample.

45. The kit of claim 44, wherein said nucleic acid isolated is mRNA.

46. The kit of claim 44, wherein said nucleic acid isolated is DNA.
